(19) United States
(12) Patent Application Publication
(10) Pub. No.: US 2005/0009112 A1
(43) Pub. Date: Jan. 13, 2005

(54) Methods for identifying Rheb effectors as lead compounds for drug development for diabetes and diseases associated with abnormal cell growth

(75) Inventors: Bruce A. Edgar, Seattle, WA (US); Leslie J. Saucedo, Seattle, WA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Fred Hutchinson Cancer Research Center, Office of Technology Transfer, Seattle, WA (US)

(21) Appl. No.: 10/796,905

(22) Filed: Mar. 8, 2004

Related U.S. Application Data
(60) Provisional application No. 60/452,919, filed on Mar. 7, 2003.

(51) Int. Cl. G01N 33/53; G01N 33/567

(52) U.S. Cl. 435/7.2

(57) ABSTRACT

Methods for identifying Rheb effectors are provided. The Rheb effectors can be Rheb agonists or antagonists and can be utilized as lead compounds for the development of drugs for the treatment of diabetes or diseases associated with abnormal cell growth. Non-human, transgenic animals over-expressing Rheb protein, and methods of making such transgenic animals, are also provided.

### CONTINUITY

This application claims the benefit of U.S. Provisional Patent Application No. 60/452,919, filed Mar. 7, 2003, the disclosure of which is incorporated by reference herein.

### STATEMENT AS TO RIGHTS TO INVENTIONS MADE UNDER FEDERALLY SPONSORED RESEARCH AND DEVELOPMENT

This work was supported by grants from the National Institutes of Health GM20590 and GM51186. The Federal Government may have certain rights in this invention.

### BACKGROUND OF THE INVENTION

Diabetes mellitus is a syndrome with interrelated metabolic, vascular, and neuropathic components. The metabolic component, generally characterized by hyperglycemia, comprises alterations in carbohydrate, fat and protein metabolism caused by reduced insulin secretion and/or ineffective insulin action. Generally, there are two types of diabetes mellitus: type I and type II. Type I diabetes, “insulin-dependent” diabetes, is characterized by an inability to synthesize insulin. Type II diabetes, “non-insulin-dependent” diabetes, is characterized by an ability to synthesize insulin, but this insulin is either insufficient for the needs of the subject, or is not effectively used by the subject.

Type I diabetes is an autoimmune disease, in which the body's islet cells are destroyed by the body's own immune system. Type II diabetes appears to be a metabolic disorder resulting from the body's inability either to make a sufficient amount of insulin or to properly use the insulin that is produced. Insulin secretion and insulin resistance are considered the major metabolic defects, but the precise genetic factors involved remain unknown. Type I diabetes is treated by insulin injection, and type II diabetes is typically treated by administration of drugs, such as an oral hypoglycemic (e.g., tolbutamide or glipizide) or thiazolidinedione (e.g., glitazone), insulin (which results in insulin levels which are sufficient to stimulate insulin-resistant tissues), an immunomodulatory drug, and the like. Such treatments can be ineffective, however, due to side-effects, increased insulin resistance, or the like.

One of the obstacles to the development of new treatments for diabetes, and for other diseases, such as cancer, is a lack of understanding of the interacting members of cellular pathways. For example, in the insulin response pathway, there has been a lack of understanding of the insulin/PI3K signaling pathway. Similarly, the pathways involved in cancer or other hyperproliferative disease are not completely understood.

Rheb was originally identified as a Ras homologue enriched in brain but is also expressed in many other tissues. (See, e.g., Yamagata et al., J. Biol. Chem. 269:16333-39 (1994); Gromov et al., FEBS Lett. 377:221-26 (1995); Clark et al., J. Biol. Chem. 272:10608-15 (1997).) Rheb contains arginine and serine residues at amino acids 15 and 16 that are homologous to amino acid residues 12 and 13 of Ras. In Ras, substitution of amino acids 12 and 13 confers GTPase insensitivity and constitutive activity. These amino acid substitutions in Ras suggest that Rheb would be constitutively bound to GTP and, thus, active. Recently, Im et al. (Oncogene 21:6356-65 (2002)) demonstrated that in three different mammalian cell lines, Rheb exists in a highly activated state and that the relative amount of Rheb bound to GTP does not substantially increase upon serum stimulation. The high percentage of Rheb bound to GTP was maintained even after substitution of the amino acid residues at position 15 and 163, suggesting that the high activation state of Rheb may not be intrinsic, but rather reflects an excess of activating proteins-guanine nucleotide exchange factors (GEFs).

The idea that Rheb may also be regulated transcriptionally is supported by the rapid induction of rheb mRNA following both neuronal stimulation in animals and growth factor/serum stimulation in tissue culture (see, e.g., Yamagata, K. et al., supra). In spite of Rheb's responsiveness to growth factors at the level of transcription, stable transfection of Rheb into cultured mammalian cells failed to accelerate growth rates or lead to transformation. (See, e.g., Yee and Worley, Mol. Cell Biol. 17:921-33 (1997); Clark et al., supra.)

Rheb protein has been demonstrated to bind to Raf1 in vitro and B-Raf in vivo. (See, e.g., Im et al., supra; Yee and Worley, supra; Clark et al., supra.) Both are effectors of Ras signaling and exogenous over-expression of Rheb may antagonize Ras in some situations (see, e.g., Clark et al., supra). However, epistasis tests in yeast have found no overlap between endogenous Ras and Rheb function (see, e.g., Mach et al., Genetics 155:611-22 (2000)). Instead, Ras and Rheb were reported to have different functions.

Rheb has been shown to have a nutrient sensing role in fungi, a unique function for a member of the Ras superfamily. (See, e.g., Mach et al., supra; Panepinto et al., Fungal Genet Biol. 36:207-14 (2002).) In S. pombe, reduced levels of Rheb result in a premature growth arrest in response to decreased levels of nitrogen (see, e.g., Mach et al., supra). Additionally, in A. fumigatus, transcription of Rheb is induced following nitrogen starvation, though a similar induction is not seen in S. pombe (see, e.g., Mach et al., supra; Panepinto et al., supra). In S. cerevisiae, Rheb also appears to have a direct role in regulating nutrient import because mutations of rheb resulted in increased uptake of arginine and lysine (see, e.g., Urano et al., J. Biol. Chem. 275:11198-206 (2000)). The comparable role of Rheb in higher eukaryotes in nutrient sensing has not hitherto been appreciated.

### BRIEF SUMMARY OF THE INVENTION

The present invention provides methods for identifying candidate compounds that are Rheb effectors. Rheb effectors are useful for the regulation of plasma glucose levels (e.g., glucose uptake and/or utilization), the regulation of abnormal cell growth (e.g., obesity, tuberous sclerosis, and certain cancers), and other processes mediated by Rheb.

In one aspect, methods are provided for identifying a lead compound for diabetes drug development. The methods generally include contacting a first aliquot of cells expressing a Rheb protein with a candidate compound under suitable conditions and for a period of time sufficient to affect Rheb activity, and measuring a parameter of the first aliquot of cells. The parameter is associated with Rheb activity. The parameter also can be measured in a second aliquot of control cells (e.g., cells not contacted with the compound, or cells contacted with a different compound or with an inert compound). The measured parameters of the first and second aliquots of cells are compared. A change in the parameter is associated with an increase in Rheb activity, and indicates that the compound affects Rheb activity. The detected or identified candidate compound optionally can be used as a lead compound for diabetes drug development.

In certain embodiments, the Rheb protein can be over-expressed, and the measured parameter can be, for example, cell size, cell viability, glucose uptake or utilization, Rheb-GTP levels, or the like. The Rheb protein can be, for example, human or Drosophila Rheb protein.

In another aspect, methods for identifying a lead compound for diabetes drug development are provided. The methods generally include: (1) contacting a candidate compound with Rheb protein under conditions conducive to binding of the compound to the Rheb protein; and (2) detecting a resulting candidate compound/Rheb protein complex, where the candidate compound increase (e.g., stimulates) or decreases Rheb activity. The detected compound optionally can be used as a lead compound for diabetes drug development. The Rheb protein can be, for example, human or Drosophila Rheb protein. In an exemplary embodiment, the Rheb protein is human Rheb protein expressed in Drosophila cells.

In certain embodiments, contacting of the candidate compound with the Rheb protein is performed with cultured cells (e.g., human, Drosophila or mammalian cells), and the stimulation of Rheb activity is detected, for example, by detecting an increase in cell size or a prolongation of cell viability. The Rheb protein can be over-expressed in the cultured cells. In other embodiments, the Rheb protein is contacted with the candidate compound in Drosophila larvae, or by administration of the candidate compound to Drosophila during eye development. Stimulation of Rheb activity can be detected, for example, by an enlarged eye phenotype, by changes in Rheb-GTP binding or Rheb-mediated GTPase activity, glucose uptake or utilization, or the like.

In another aspect, methods are provided for screening a library of candidate compounds to identify a lead compound for diabetes drug development. The methods typically include contacting the candidate compounds with cells expressing a Rheb protein under suitable conditions and for a period of time sufficient to affect Rheb activity. A parameter of the contacted cells is measured for a change in phenotype associated with Rheb agonist activity. The change in the parameter is used to determined whether the candidate compound stimulates Rheb activity to identify a Rheb agonist. The measured parameter can be, for example, cell size or cell viability, the size or shape of the eye in Drosophila, or glucose uptake or utilization. In certain embodiments, the Rheb protein can be over-expressed. The identified Rheb agonist can optionally be used as a lead compound for diabetes drug development.

In yet another aspect, methods are also provided for identifying a lead compound for drug development for a disease associated with abnormal cell growth. The methods generally include contacting a first aliquot of cells expressing a Rheb protein with a candidate compound under suitable conditions and for a period of time sufficient to affect Rheb activity and measuring a parameter of the first aliquot of cells associated with Rheb activity. The parameter can optionally be measured in a second aliquot of control cells. The measured parameter of the cells can be compared, where a change in the parameter is associated with a change in Rheb activity. For example, the candidate compound can inhibit Rheb activity. The Rheb protein can be, for example, human or Drosophila Rheb protein. The candidate compound can optionally be used as a lead compound for drug development for the disease associated with abnormal cell growth. The measured parameter can be, for example, cell size, glucose uptake or utilization, or the like.

In a related aspect, methods are provided for screening a library of candidate compounds to identify a lead compound(s) for drug development for a disease associated with abnormal cell growth. The methods generally include contacting the candidate compounds with cells expressing a Rheb protein under suitable conditions and for a period of time sufficient to affect Rheb activity and measuring a parameter of the contacted cells for a change in phenotype associated with Rheb antagonist activity. The measured parameter can be used to determine whether the candidate compound inhibits Rheb activity to identify a Rheb antagonist. The identified candidate compound can optionally be used as a lead compound for drug development for a disease associated with abnormal cell growth.

Non-human, transgenic animals over-expressing Rheb protein are also provided. In one aspect, the transgenic animal typically has increased cell or organ size as compared with an animal not over-expressing Rheb protein. The transgenic animal can, for example, over-express human or Drosophila Rheb protein. The transgenic animal can be, for example, a primate, mammal, bovine, porcine, ovine, equine, avian, rodent, fowl, piscine, or crustacean. In a specific embodiment, the animal is a farm animal, such as, for example, a chicken, cow, bull, horse, pig, sheep, goose or duck.

In another aspect, the transgenic, non-human animal over-expresses Rheb protein, and the over-expression results in increased size or growth rate of the animal. In yet another aspect, methods are provided for increasing the size or growth rate of a non-human, transgenic animal. Such methods generally include stably introducing into a genome of an animal cell a Rheb gene, whereby Rheb protein is over-expressed; and producing a non-human transgenic animal from the animal cell. In another aspect, methods are provided for increasing the size or growth rate of a non-human, transgenic animal. The methods generally include stably introducing into a genome of an animal cell a Rheb gene, whereby Rheb protein is over-expressed; and producing an animal from the animal cell.

These and other embodiments are exemplified in the following description and drawings.

### BRIEF DESCRIPTION OF THE DRAWINGS

FIGS. 1a-d. Rheb is a regulator of growth. FIG. 1a: Expression of Rheb from GSjE2 was induced using gmrGAL4 and tissue growth examined in adult eyes of females using SEM. Control animals contain gmrGAL4 alone. FIG. 1b: The polymerase chain reaction, using genomic DNA as a substrate, was used to map the position of GSjE2 (indicated by the open arrowhead) and rheb^{PΔ1} and rheb^{PΔ2}. The boundary of the deletions are denoted by numbers corresponding to Genbank accession # AE003602.3. FIG. 1c: Northern analysis to detect expression of Rheb mRNA in imprecise excision lines rheb^{PΔ1} and rheb^{PΔ2}. Rp49 is used as a loading reference. FIG. 1d: Animals transheterozygous (rheb^{PΔ1/PΔ2}) for loss of rheb (middle) and their heterozygous siblings (rheb^{PΔ1 or PΔ2}/TM3GFP, top) were photographed every 24 hours throughout larval development. A partial rescue of the growth inhibition was seen when hsGAL4 and UAS-Rheb transgenes were introduced into the rheb^{PΔ1/PΔ2} animals (bottom).

FIGS. 2a-b. Rheb increases the size of wing and fat body cells. FIG. 2a: Induction of UASRheb with enGAL4 induces overgrowth in the posterior compartment (p) of the adult wing (11% larger). Increased distance between wing hairs (upper, right inset) indicates that wing cells are enlarged when Rheb is overexpressed. Animals are female and control animal expresses enGAL4 alone. FIG. 2b: Clones of cells over-expressing Rheb and GFP under the control of actGAL4 were induced in fat body tissue prior to endoreduplication. GFP expression (top) and DNA staining (Hoechst 33258, bottom) are shown for identical sections. The control animal expresses GFP alone.

FIG. 3. Rheb alters cell cycle phasing but does not affect the rate of cell division. Flow cytometry was performed on dissociated wing disc cells containing clones of cells over-expressing GFP (control, left panels) or Rheb and GFP (+Rheb, right panels). Hoechst 33342 was used to assess DNA content (top) and forward scatter was used to quantify cell size (bottom). Cells over-expressing GFP are indicated by the gray fill. Cells that do not express transgenes serve as internal controls for each sample and are indicated by the black line.

FIG. 4. Genetic interactions between Rheb and PTEN, TSC1/2, or S6k. GmrGAL4 was used to drive expression of Rheb in post-mitotic cells of the eye. The ability of Rheb to promote overgrowth in the eye tissue of animals over-expressing PTEN, co-over-expressing TSC1 and TSC2, or lacking S6k was examined using SEM. All animals are females and control animal contains gmrGAL4 alone.

FIG. 5. The reduction of cell size resulting from loss of tor is dominant over the ability of Rheb to promote cellular growth. Flow cytometry was performed on dissociated wing discs which contained clones of cells over-expressing Rheb (+Rheb), lacking tor ( tor^{ΔP}/tor^{ΔP}), or both (tor^{ΔP}/tor^{ΔP}, +Rheb). Hoechst 33342 was used to assess DNA content (top) and forward scatter was used to quantify cell size (bottom). The experimental populations co-express GFP and are indicated by the gray fill. Non-experimental cells from the same tissues are indicated by the black line. The control expresses GFP only.

FIGS. 6a-c. Rheb regulates TOR/S6K signaling in Drosophila cells. FIG. 6a: HA-S6K was transfected into S2 cells in the presence or absence of myc-Rheb. HA-S6K was immunoprecipitated from cell lysates and probed with anti-phospho-Thr398 S6K (upper gel) or anti-HA (middle gel). A portion of the cell lysate was directly probed with anti-myc (lower gel). FIG. 6b: S2 cells were transfected with or without myc-Rheb and incubated in culture media with or without amino acids, as indicated. Cell lysates were probed with anti-phospho-Thr398 S6K (upper gel), anti-S6K (middle gel) and anti-myc (lower gel). FIG. 6c: S2 cells treated with control or indicated dsRNA were incubated in complete or amino acid-free medium for 2 hours. Cell lysates were probed with anti-phospho-Thr398 S6K (upper gel), anti-S6K (middle gel) and TSC2 (lower gel).

FIG. 7. Over-expression of Rheb, but not S6K, promotes growth in the absence of nutrients. The effect of Rheb or S6K over-expression in fat body tissue was examined in larvae following 3 days of a protein-free diet. DNA was stained with Hoechst 33258 and cells over-expressing Rheb (top panels) or S6K (bottom panels) were co-expressing GFP.

### DETAILED DESCRIPTION

The present invention provides methods of identifying candidate compounds that are Rheb effectors. Rheb effectors are useful for the regulation of plasma glucose levels (e.g., glucose uptake and/or utilization) as well as regulation of abnormal cell growth (e.g., obesity, tuberous sclerosis, and certain cancers). Candidate compounds identified as Rheb effector can be used as lead compounds for the development of therapeutic agents for the treatment of diseases or disorders associated with plasma glucose levels (e.g., glucose uptake and/or utilization), abnormal cell growth, or the like. In certain embodiments, the disease or disorder associated with regulation of plasma glucose levels is diabetes, such as Type I or Type II diabetes. In other embodiments, the disease or disorder is associated with abnormal cell growth, such as, for example, those associated with hyperactivation of insulin/PI3K signaling pathway.

Rheb functions as a regulator of cell growth and interacts with components of the insulin/PI3K and TOR signaling pathways. Rheb over-expression phenotypes most closely resemble those caused by hyperactivation of insulin/PI3K signaling. Rheb-induced overgrowth can bypass two negative regulators in this pathway, PTEN and TSC1/2, suggesting that Rheb acts further downstream. TOR is epistatic to overexpressed Rheb, indicating that Rheb induces cell growth either as a downstream component of insulin/PI3K signaling or in a parallel pathway that requires TOR. Rheb-mediated cell growth requires TOR, placing Rheb between TSC1/2 and TOR and thus as a downstream effector of insulin/PI3K signaling and nutrient sensing.

In one aspect, methods are provided to identify Rheb effectors. These methods generally include contacting Rheb protein, or cells expressing Rheb protein, with a candidate compound and determining whether the candidate compound affects Rheb activity. As used herein, a “candidate compound” refers to a molecule that is amenable to a screening technique. Suitable candidate compounds can be proteins, polypeptides, peptides and small molecules. A “small molecule” refers to a non-protein-based moiety.

Rheb effectors can affect rheb gene transcription, rheb RNA processing, Rheb protein synthesis, and/or Rheb protein modification, activity, stability and/or localization. For example, with regard to Rheb protein activity, effectors can affect Rheb GTP-binding or GTPase activity by, e.g., binding to a site within the GTPase active site, binding to an allosteric site that affects GTPase activity, or blocking the association of Rheb with the GTPase Activating Proteins (GAPs) (e.g., the GAP domain of TSC2). Also, in the case of Rheb localization, effectors can, for example, affect the farnesylation of Rheb protein required for membrane anchorage and activity. Rheb effectors can be utilized, for example to modify cell proliferation, glucose uptake or utilization, amino acid uptake and/or utilization, and/or metabolism.

In certain embodiments, a Rheb effector can be an antagonist of Rheb. Methods are provided for identifying candidate compounds that specifically inhibit the activity or expression of Rheb nucleic acids or Rheb proteins. As used herein, an “antagonist” refers to a moiety that inhibits the activity of Rheb by affects on rheb gene transcription, rheb RNA processing, Rheb protein synthesis, and/or Rheb protein modification, activity, stability and/or localization. “Inhibit” or “inhibiting,” refer to a response that is decreased or prevented in the presence of a compound as compared to a response in the absence of the compound. For example, a Rheb protein antagonist can inhibit the intracellular response when it binds to Rheb protein, as compared to a cell not contacted with the Rheb antagonist (e.g., a control cell).

In other embodiments, a Rheb effector can be an agonist of Rheb. Methods are provided for identifying candidate compounds that specifically stimulate the activity or expression of Rheb nucleic acids or Rheb protein. As used herein, an “agonist” refers to a moiety that stimulates the activity of Rheb. For example, a Rheb protein agonist can stimulate an intracellular response when it binds to Rheb protein, as compared to a cell not contacted with the Rheb agonist.

In another aspect, methods for identifying candidate compounds that specifically bind to Rheb protein are provided. Rheb effectors can be identified by in vivo, ex vivo and/or in vitro assays. In certain embodiments, a detected Rheb protein effector can be used as a lead compound for drug development.

Rheb protein can be from any suitable animal or vertebrate source, such as, for example human Rheb. In one embodiment, the human Rheb protein has the amino acid sequence reported in Genbank Accession No. Z29677 or NP_{—}005605 (the disclosures of which are incorporated by reference herein). (See also Genbank Accession Numbers AAH66307, AAH16155 and Q15382.) In other embodiments, the Rheb protein is from a non-human source, such as, for example, primates, rodents (e.g., mouse or rat), Drosophila, and the like. In certain specific embodiments, the Rheb protein has an amino acid sequence associated with Unigene Cluster Mm.259708 (formerly Mm.68190) or Hs.159013, such as, for example, Accession No. pir:S68410, pir:S68419, NP_{—}444305.1, pir:155401, sp:Q9VND8, or the like (which are incorporated by reference herein).

Rheb protein also include “functionally active” Rheb polypeptides having one or more functional activities associated with a full-length (wild-type) Rheb protein (e.g., GTP-binding, GTPase activity, and the like). Functionally active Rheb protein include Rheb polypeptides, fragments, derivatives and analogs thereof.

Rheb nucleic acids include nucleic acids encoding Rheb protein, such as, for examples, those set forth above. The terms “polynucleotide” and “nucleic acid” refer to a polymer composed of a multiplicity of nucleotide units (ribonucleotide or deoxyribonucleotide or related structural variants) linked via phosphodiester bonds. Polynucleotides and nucleic acids include RNA, cDNA, genomic DNA, synthetic forms, and mixed polymers, both sense and antisense strands, and can also be chemically or biochemically modified or can contain non-natural or derivatized nucleotide bases, as will be readily appreciated by the skilled artisan. Rheb nucleic acids typically encode a Rheb protein or functionally active Rheb polypeptide, fragments, derivative or analogs.

In another aspect, methods are provided to identify Rheb effectors by screening candidate compounds in vivo for those that affect Rheb activity. As will be appreciated by the skilled artisan, Rheb effectors can be identified during large-scale screening, wherein the identity of each compound is known during the screening process. Alternatively, Rheb effectors can be identified during large-scale screening, wherein the identity of each compound is not known during the screening process.

As used herein, “identify” refers to the determination of a candidate compound as a Rheb effector (e.g., either an agonist or antagonist), whether or not the specific identity or chemical structure of that compound is known. “Detect” or “identify” can be synonyms, according to context.

Drosophila, yeast or other animal systems can be used to screen candidate compounds for Rheb effectors. In certain embodiments, the endogenous Rheb protein can be overexpressed, such as, for example, by introducing additional copies of a Rheb nucleic acid or expression construct encoding a Rheb protein. In other embodiments, the endogenous Rheb gene can be inactivated or deleted and replaced with a heterologous Rheb gene (such as the cDNA). For example, the endogenous Drosophila or yeast gene(s) can be replaced with a human Rheb gene or cDNA in Drosophila or yeast, respectively. In a related example, the endogenous Rheb gene can be inactivated and a heterologous Rheb gene introduced.

In an exemplary embodiment, Drosophila flies can be screened with candidate compounds to detect or identify those compounds that specifically suppress growth phenotypes caused by ectopic over-expression of the Rheb genes. In one example, Rheb is over-expressed in the Drosophila eye, giving a visible enlarged eye phenotype. As used herein, “over-expressed” refers to an increased Rheb protein or activity, as compared with the protein activity normal or typically present (e.g., in a cell, a tissue, an organism, or the like). Candidate compounds (e.g., potential inhibitors of Rheb) are administered to the flies (e.g., by feeding) during the stage when the eye develops, and compounds that inhibit Rheb function are detected or identified by their ability to partially or fully restore the eye to normal size and morphology.

In another example, Drosophila larvae can be contacted with candidate compounds to detect or identify those compounds that suppress the starvation-sensitivity (lethal) phenotype associated with over-expression of Rheb. Successful candidate compounds which are detected or identified are those that prolong the life of Rheb-expressing animals under starvation conditions. Such a screen can also optionally screen out compounds that are toxic. In addition, because endogenous Rheb is required for cell growth, the screen can identify compounds that selectively affect cells over-expressing Rheb protein but not cells having normal endogenous Rheb protein levels and/or activity.

In other examples, Rheb agonists can be identified in Drosophila, yeast or other suitable animal systems. For example, Drosophila flies can be screened with candidate compounds to detect or identify those compounds that specifically stimulate growth phenotypes associated with ectopic over-expression of the Rheb genes. Candidate compounds (e.g., potential Rheb agonists) are administered to the flies (e.g., by feeding) during the stage when the eye develops, and compounds that stimulate Rheb function are detected or identified by their ability to produce flies having a visibly enlarged eye phenotypes.

In yet another example, Drosophila larvae can be contacted with candidate compounds to detect or identify those compounds that enhance the starvation-sensitivity (lethal) phenotype associated with Rheb. Successful candidate compounds that are detected or identified are those that specifically decrease the life of animals under starvation conditions. Such a screen also optionally can be followed by screens to identify or eliminate compounds that are toxic.

In other exemplary embodiments, yeast systems can be used to detect or identify candidate compounds that are Rheb effectors. In an exemplary embodiment, the yeast plasmid shuffling system allows the identification of effectors that specifically affect expression or activity of a Rheb protein. In a particular embodiment, a yeast strain that has a null allele of the endogenous yeast Rheb gene is rescued by an heterologous Rheb gene or cDNA (e.g., from human, Drosophila, or the like). Such yeast strains can be contacted with candidate compounds and Rheb effectors detected or identified by examining effects of the candidate compounds on the cells (e.g., effects on viability during nutrient starvation). In a specific example, a yeast strain having a null allele of the endogenous yeast Rheb gene, and expressing either human Rheb cDNA or Drosophila Rheb cDNA, can be screened for Rheb effectors that specifically affect the human or Drosophila Rheb protein under nutrient starvation conditions. Similarly, agonists and antagonists can be identified that affect a particular allele or mutant of a Rheb nucleic acid or Rheb protein (e.g., by affecting cell growth, cell size, viability and/or cell division).

In another exemplary embodiment, a method comprises administering a candidate compound to a first cell that expresses a first Rheb protein; administering the candidate compound to a second cell that expresses a second, different Rheb protein; and determining whether the candidate compound modulates the activity of the first Rheb protein but not the activity of the second Rheb protein. For example, the first Rheb protein can be human Rheb protein, and the second can be yeast Rheb protein. Alternatively, the first Rheb protein can be a mutant, and the second Rheb protein can be wild-type.

In a typical ex vivo assay, recombinant cells expressing a Rheb protein can be used to screen candidate compounds for those that affect Rheb expression or Rheb activity. Effects on Rheb expression can include, for example, transcription of Rheb RNA, processing of Rheb RNA to mRNA, translation of Rheb mRNA, synthesis of Rheb protein, effects on Rheb protein function, and/or on Rheb protein stability or localization. Such effects on Rheb expression can be identified as physiological changes, such as, for example, changes in cell size, cell growth rate, cell division and/or cell viability. In an exemplary embodiment, candidate compounds are administered to recombinant cells over-expressing human or Drosophila Rheb protein to detect or identify those compounds that affect cell size.

A typical ex vivo assay can be performed, for example, using human, mammalian, animal or insect cells, and can be performed using isolated cells, tissues, organs, or the like. In certain embodiments, the ex vivo assay is performed in a non-yeast, eukaryotic organism.

Over-expressed Rheb protein typically increases cell size, and inhibition of this phenotype (reduction in cell size) can be used to detect or identify Rheb antagonists. Similarly, Rheb agonists can be identified as those that increase cell size. Suitable methods for monitoring cell size include, for example, photometric or flow-cytometric assays of cells (e.g., determination of forward scatter by FACS) after contacting the cells with candidate compounds (e.g., by addition to cell culture media). A reporter can optionally be included. For example, Green Fluorescence Protein (GFP) reporter can also be expressed in the cells and/or in control cells.

In another exemplary embodiment, an ex vivo cell-based starvation-sensitivity assay can be used to detect or identify candidate compounds that affect cells in culture. For example, Drosophila, yeast or human cells over-expressing Rheb can be starved for amino acids. The cells can be contacted with candidate compounds. Successful Rheb antagonist compounds are those that allow the cells to remain viable for longer time periods than cells not contacted with the candidate compounds. As will be apparent to the skilled artisan, such assays can be run in large format, or high throughput screens. For example, multi-well plates can be used and the cells screened for a scorable marker or stain for cell viability. Optionally, after detecting or identifying potential candidate compounds, the candidates can be re-screened using phospho-S6-kinase levels as a specific readout for Rheb activity in Drosophila S2 or other cells.

In another embodiment, the yeast two-hybrid system can be for used selecting interacting proteins in yeast (see, e.g., Fields and Song, Nature 340:245-46 (1989); Chien et al., Proc. Natl. Acad. Sci. USA 88:9578-82 (1991); the disclosures of which are incorporated by reference herein). For example, a fusion protein comprising human Rheb protein and a GCN4 domain can be expressed in yeast. A library of fusion proteins comprising candidate peptides, polypeptides or proteins, joined to the other GCN4 domain can be screened for those compounds that interact with the human Rheb protein. Candidate compounds identified by such a screen can be further screened for Rheb agonist or antagonist activity.

Candidate compounds also can be identified by in vitro assays. For example, recombinant cells expressing Rheb nucleic acids can be used to recombinantly produce Rheb protein for in vitro assays to identify candidate compounds that bind to Rheb protein. Candidate compounds (such as putative binding partners of Rheb or small molecules) are contacted with the Rheb protein under conditions conducive to binding, and then candidate compounds that specifically bind to the Rheb protein are identified. The Rheb protein can optionally be attached to a solid support. For example, Rheb protein can be attached to microtiter dishes via antibody linkage. Similar methods can be used to screen for candidate compounds that bind to nucleic acids encoding Rheb.

Suitable assays to detect changes in Rheb activity in in vitro, ex vivo and in vivo assays can further include, for example, monitoring Rheb protein and/or message levels. Rheb is a dose-dependent effector. Levels of Rheb protein or RNA can be measured relative to control cells to determine whether a candidate compound affects Rheb activity. For example, Rheb protein levels can be measured by immunoassay using antibody against Rheb protein. Suitable immunoassays include, for example, competitive and non-competitive assay systems using techniques such as radioimmunoassays, ELISA (enzyme linked immunosorbent assay) “sandwich” immunoassays, immunoradiometric assays, in situ immunoassays (using colloidal gold, enzyme or radioisotope labels, and the like), Western blots, immunofluorescence assays, protein A assays, immunoelectrophoresis assays, and the like. (See generally Harlow and Lane, Antibodies: A Laboratory Manual (Cold Spring Harbor Laboratory, New York, 1988); Harlow and Lane, Using Antibodies: A Laboratory Manual(Cold Spring Harbor Laboratory, New York, 1999).) Similarly, Rheb RNA levels can be measured by suitable assay, such as for example, polymerase chain reaction assay, Southern blotting, Northern blotting, or the like. (See generally Sambrook et al., Molecular Cloning: A Laboratory Manual, 3d Ed. (Cold Spring Harbor Laboratory Press, New York 2001)); Ausubel et al., Short Protocols in Molecular Biology (John Wiley & Sons, Inc., New York, 1999), the disclosures of which are incorporated by reference herein). In addition, assays can be used to detect Rheb gene amplification. Suitable assays include for example, Southern blotting, polymerase chain reaction, and the like. (See generally Sambrook et al. (supra); Ausubel et al. (supra).) In addition, Rheb activity can be measured by Rheb-GTP/Rheb-GDP ratio, where Rheb-GTP is the active form. Such assays are described, for example, in Zhang et al. (Nat. Cell Biol. 5:578-81 (2003); the disclosure of which is incorporated by reference herein).

Candidate compounds can be obtained from any suitable source. Many libraries are known in the art, such as, for example, chemically synthesized libraries, recombinant phage display libraries, and in vitro translation-based libraries. In addition, natural product libraries can be used as a source of candidate compounds. Similarly, diversity libraries, such as random or combinatorial peptide or non-peptide libraries can be used. Methods of preparing candidate compounds are known in the art, and include, for example, diversity libraries, such as random or combinatorial peptide or non-peptide libraries.

Examples of chemically synthesized libraries are described by Fodor et al. (Science 251:767-73 (1991)), Houghten et al. (Nature 354:84-86 (1991)), Lam et al. (Nature 354:82-84 (1991)), Medynski (Bio/Technology 12:709-10 (1994)), Gallop et al. (J. Med. Chem. 37:1233-51 (1994)), Ohlmeyer et al. (Proc. Natl. Acad. Sci. USA 90:10922-26 (1993)), Erb et al. (Proc. Natl. Acad. Sci. USA 91:11422-26 (1994)), Houghten et al. (Biotechniques 13:412-21 (1992)), Jayawickreme et al. (Proc. Natl. Acad. Sci. USA 91:1614-18 (1994)), Salmon et al. (Proc. Natl. Acad. Sci. USA 90:11708-12 (1993)), International Patent Publication WO 93/20242, and Brenner and Lerner (Proc. Natl. Acad. Sci. USA 89:5381-83 (1992)).

Examples of phage display libraries are described in Scott and Smith (Science 249:386-90 (1990)), Devlin et al. (Science 249:404-06 (1990)), Christian et al. (J. Mol. Biol. 227:711-18 (1992)), Lenstra (J. Immunol. Meth. 152:149-57 (1992)), Kay et al. (Gene 128:59-65 (1993)), and International Patent Publication WO 94/18318.

In vitro translation-based libraries include, but are not limited to, those described in International Patent Publication WO 91/05058, and Mattheakis et al. (Proc. Natl. Acad. Sci. USA 91:9022-26 (1994)). By way of examples of nonpeptide libraries, a benzodiazepine library (see, e.g., Bunin et al., Proc. Natl. Acad. Sci. USA 91:4708-12 (1994)) can be adapted for use. Peptide libraries (see, e.g., Simon et al., Proc. Natl. Acad. Sci. USA 89:9367-71(1992)) also can be used. Another example of a library that can be used, in which the amide functionalities in peptides have been permethylated to generate a chemically transformed combinatorial library, is described by Ostresh et al. (Proc. Natl. Acad. Sci. USA 91:11138-42 (1994)).

Screening of the libraries can be accomplished by any of a variety of commonly known methods. For example, the following references disclose screening of peptide libraries: Parmley and Smith (Adv. Exp. Med. Biol. 251:215-18 (1989)); Scott and Smith (supra); Fowlkes et al. (BioTechniques 13:422-28 (1992)); Oldenburg et al. (Proc. Natl. Acad. Sci. USA 89:5393-97 (1992)); Yu et al. (Cell 76:933-45 (1994)); Staudt et al. (Science 241:577-80 (1988)); Bock et al. (Nature 355:564-66 (1992)); Tuerk et al. (Proc. Natl. Acad. Sci. USA 89:6988-92 (1992)); Ellington et al. (Nature 355:850-52 (1992)); U.S. Pat. Nos. 5,096,815; 5,223,409 and 5,198,346; Rebar and Pabo (Science 263:671-73 (1994)); and International Patent Publication WO 94/18318.

In a specific embodiment, screening can be carried out by contacting the library members with a Rheb protein (or a Rheb nucleic acid or derivative) immobilized on a solid phase and harvesting those library members that bind to the polypeptide (or nucleic acid or derivative). Examples of such screening methods, termed “panning” techniques, are described by way of example in Parmley and Smith (Gene 73:305-18 (1988)); Fowlkes et al. (supra); International Patent Publication WO 94/18318; and in references cited hereinabove.

In another aspect, transgenic animals over-expressing one or more Rheb genes, and methods of making such animals, are provided. As used herein, the term “transgenic animal” refers to a non-human animal that harbors cells that over-express one or more Rheb genes. A transgenic animal can be, for example, a primate, mammal, avian, porcine, ovine, bovine, feline, canine, fowl, rodent, fish, insect, crustacean, and the like. In specific embodiments, the transgenic animal can be a sheep, goat, horse, cow, bull, pig, rabbit, guinea pig, hamster, rat, gerbil, mouse, chicken, ostrich, emu, turkey, duck, goose, quail, parrot, parakeet, cockatoo, cockatiel, trout, cod, salmon, crab, king crab, lobster, shrimp or Drosophila. Transgenic animals include chimeric animals (i.e., those composed of a mixture of genetically different cells), mosaic animals (i.e., an animal composed of two or more cell lines of different genetic origin or chromosomal constitution, both cell lines derived from the same zygote), immature animals, fetuses, blastulas, and the like.

A Rheb gene can be a homologous or heterologous Rheb gene, a homologous or heterologous Rheb cDNA, or an expression construct comprising a promoter, an open reading frame encoding a Rheb protein and other elements necessary for expression of the Rheb protein. As used herein, a “homologous” refers to nucleic acid from the same species or subspecies. “Heterologous” refers to a nucleic acid from a different species or subspecies.

In transgenic animals, over-expression of the Rheb gene causes an increased size of at least a portion of the animal, as compared with wild-type, non-transgenic animal (i.e., not over-expressing a Rheb gene). In certain embodiments, the transgenic animals have enlarged tissues that contain more cells or larger cells than tissues from a non-transgenic animal. Transgenic animals can contain one or more over-expressed Rheb genes, which can be located at the endogenous Rheb locus, and/or at a non-Rheb locus (or loci).

Transgenic, non-human animals over-expressing a Rheb gene can be prepared by methods known in the art. In general, a Rheb gene is introduced into target cells, which are then used to prepare a transgenic animal. Rheb genes can be introduced into target cells, such as for example, pluripotent or totipotent cells such as embryonic stem (ES) cells (e.g., murine embryonal stem cells or human embryonic stem cells) or other stem cells (e.g., adult stem cells); germ cells (e.g., primordial germ cells, oocytes, eggs, spermatocytes, or sperm cells); fertilized eggs; zygotes; blastomeres; and the like; fetal or adult somatic cells (either differentiated or undifferentiated); and the like. In certain embodiments, the Rheb gene can be introduced into embryonic stem cells or germ cells of animals (e.g., mammals, farm animals, livestock, hatchery animals, and the like) to prepare a Rheb transgenic animal.

Embryonic stem cells can be manipulated according to published procedures (see, e.g., Teratocarcinomas and Embryonic Stem Cells: A Practical Approach, Robertson (ed.), IRL Press, Washington, D.C. (1987); Zjilstra et al., Nature 342:435-38 (1989); Schwartzberg et al., Science 246:799-803 (1989); U.S. Pat. Nos. 6,194,635; 6,107,543; and 5,994,619; each of which is incorporated herein by reference in their entirety). Methods for isolating primordial germ cells are well known in the art. For example, methods of isolating primordial germ cells from ungulates are disclosed in U.S. Pat. No. 6,194,635 (the disclosure of which is incorporated by reference herein in its entirety). Briefly, primordial germ cells are isolated from gonadal ridges of an embryo at a particular stage in development (e.g., day-25 porcine embryos or day 34-40 bovine embryos). The stage of development at which primordial germ cells are extracted from an embryo of a particular species will vary with the species, as will be appreciated by the skilled artisan. Determination of the appropriate embryonic developmental stage for such extraction is readily performed using the guidance provided herein and ordinary skill in the art.

Primordial germ cells can be isolated from the dorsal mesentery and usually test positive for alkaline phosphate activity. The cells can be isolated at a suitable time after fertilization. To ascertain that harvested cells are of an appropriate developmental age, harvested cells can be tested for morphological criteria which can be used to identify primordial germ cells which are pluripotent (see, e.g., DeFelici and McLaren, Exp. Cell Res. 142:476-82 (1982)). To further substantiate pluripotency, a sample of the extracted cells can be subsequently tested for alkaline phosphatase (AP) activity. Pluripotent cells, such as primordial germ cells, can share markers typically found on stem cells. Primordial or embryonic germ cells typically manifest alkaline phosphatase (AP) activity, and AP positive cells are typically germ cells. AP activity is rapidly lost with differentiation of embryonic germ cells in vitro. Expression of AP also has been demonstrated in ES and ES-like cells in the mouse (see, e.g., Wobus et al., Exp. Cell. Res. 152:212-19 (1984); Pease et al., Dev. Bio. 141:344-52 (1990)), rat (see, e.g., Ouhibi et al., Mol. Repro. Dev. 40:311-24 (1995)), pig (see, e.g., Talbot et al., Mol. Repro. Dev. 36:139-47 (1993)) and bovine animals (see, e.g., Talbot et al., Mol. Repro. Dev. 42:35-52 (1995)). AP activity has also been detected in murine primordial germ cell (see, e.g., Chiquoine, Anat. Rec. 118:135-46 (1954)), murine embryonic germ cells (see, e.g., Matsui et al., Cell 70:841-47 (1992); Resnick et al., Nature 359:550-51 (1992)) and porcine primordial germ cells.

In an embodiment, transgenic avian animals can be prepared using avian primordial germ cells. Such methods are disclosed, for example, in U.S. Pat. No. 5,156,569 (the disclosure of which is incorporated by reference herein in its entirety). Generally, primordial germ cells are isolated and cultured in the presence of growth factors, such as, for example, leukemia inhibiting factor (LIF), stem cell factor (SCF), insulin-like growth factor (IGF) and/or basic fibroblast growth factor (bFGF).

Rheb genes can be introduced into target cells by any suitable method. For example, a Rheb gene(s) can be introduced into a cell by transfection (e.g., calcium phosphate or DEAE-dextran mediated transfection), lipofection, electroporation, microinjection (e.g., by direct injection of naked DNA), biolistics, infection with a viral vector containing a Rheb gene, cell fusion, chromosome-mediated gene transfer, microcell-mediated gene transfer, nuclear transfer, and the like.

In certain embodiments, a Rheb gene is introduced into target cells by transfection or lipofection. Suitable agents for transfection or lipofection include, for example, calcium phosphate, DEAE dextran, lipofectin, lipfectamine, DIMRIE C, Superfect, and Effectin (Qiagen), unifectin, maxifectin, DOTMA, DOGS (Transfectam; dioctadecylamidoglycylspermine), DOPE (1,2-dioleoyl-sn-glycero-3-phosphoethanolamine), DOTAP (1,2-dioleoyl-3-trimethylammonium propane), DDAB (dimethyl dioctadecylammonium bromide), DHDEAB (N,N-di-n-hexadecyl-N,N-dihydroxyethyl ammonium bromide), HDEAB (N-n-hexadecyl-N,N-dihydroxyethylammonium bromide), polybrene, poly(ethylenimine) (PEI), and the like. (See, e.g., Banerjee et al., Med. Chem. 42:4292-99 (1999); Godbey et al., Gene Ther. 6:1380-88 (1999); Kichler et al., Gene Ther. 5:855-60 (1998); Birchaa et al., J. Pharm. 183:195-207 (1999); each incorporated by reference herein in its entirety.)

For avian species, which form a shell, the optimal time to introduce a Rheb gene, into avian cells is after oviposition and within six hours of activation (post-incubation) so that the cells have started to grow but have not undergone a cell division. Oviposition is the time at which the egg is laid. In the chicken, oviposition typically occurs at about 20 hours of uterine age. Rheb genes can be introduced into the blastoderm or germinal disc after oviposition, but before incubation of the egg (i.e., before the first cell division after the egg is incubated). The germinal disc is distinguished from the germinal crescent region in that the germinal disc contains undifferentiated blastodermal cells, whereas the germinal crescent region appears in the early stages of chick embryo development.

The Rheb gene(s) also can be introduced into cells by electroporation (see, e.g., Wong and Neumann, Biochem. Biophys. Res. Commun. 107:584-87 (1982)) and biolistics (e.g., a gene gun; Johnston and Tang, Methods Cell Biol. 43 Pt A:353-65 (1994); Fynan et al., Proc. Natl. Acad. Sci. USA 90:11478-82 (1993)).

Methods of introducing the Rheb gene(s) into target cells further include microinjection of the gene into target cells. For example, a Rheb gene can be microinjected into pronuclei of fertilized oocytes or the nuclei of ES cells. A typical method is microinjection of the fertilized oocyte. The fertilized oocytes are microinjected with nucleic acids encoding Rheb genes by standard techniques. The microinjected oocytes are typically cultured in vitro until a “pre-implantation embryo” is obtained. Such a pre-implantation embryo typically contains approximately 16 to 150 cells. The 16 to 32 cell stage of an embryo is commonly referred to as a “morula.” Those pre-implantation embryos containing more than 32 cells are commonly referred to as “blastocysts.” They are generally characterized as demonstrating the development of a blastocoel cavity typically at the 64 cell stage. Methods for culturing fertilized oocytes to the pre-implantation stage include those described by Gordon et al. (Methods in Enzymology 101:414 (1984)); Hogan et al. (in Manipulating the Mouse Embryo, Cold Spring Harbor Laboratory Press, Cold Spring Harbor, N.Y. (1986)); Hammer et al. (Nature 315:680 (1986)); Gandolfi et al. (J. Reprod. Fert. 81:23-28 (1987)); Rexroad et al. (J. Anim. Sci. 66:947-53 (1988)); Eyestone et al. (J. Reprod. Fert. 85:715-20 (1989)); Camous et al. (J Reprod. Fert. 72:779-85 (1989)); and Heyman et al. (Theriogenology 27:5968 (1989)) for mice, rabbits, pigs, cows, and the like. (These references are incorporated herein in their entirety.) Such pre-implantation embryos can be thereafter transferred to an appropriate (e.g., pseudopregnant) female by standard methods. Depending upon the stage of development when the Rheb gene, or the Rheb gene-containing cell is introduced into the embryo, a chimeric or mosaic animal can result. As is well known, mosaic and chimeric animals can be bred to form true germline Rheb transgenic animals by selective breeding methods well-known in the art. Alternatively, microinjected or transfected embryonic stem cells can be injected into appropriate blastocysts and then the blastocysts are implanted into the appropriate foster females (e.g., pseudopregnant females).

A Rheb gene also can be introduced into cells by infection of cells or into cells of a zygote with an infectious virus containing the gene. Suitable viruses include retroviruses (see generally Jaenisch, Proc. Natl. Acad. Sci. USA 73:1260-64 (1976)); defective or attenuated retroviral vectors (see, e.g., U.S. Pat. No. 4,980,286; Miller et al., Meth. Enzymol. 217:581-99 (1993); Boesen et al., Biotherapy 6:291-302 (1994); these references are incorporated herein in their entirety), lentiviral vectors (see, e.g., Naldini et al., Science 272:263-67 (1996), incorporated by reference herein in its entirety), adenoviruses or adeno-associated virus (AAV) (see, e.g., Ali et al., Gene Therapy 1:367-84 (1994); U.S. Pat. Nos. 4,797,368 and 5,139,941; Walsh et al., Proc. Soc. Exp. Biol. Med. 204:289-300 (1993); Grimm et al., Human Gene Therapy 10:2445-50 (1999); the disclosures of which are incorporated by reference herein in their entirety).

Viral vectors can be introduced into, for example, embryonic stem cells, primordial germ cells, oocytes, eggs, spermatocytes, sperm cells, fertilized eggs, zygotes, blastomeres, or any other suitable target cell. In an exemplary embodiment, retroviral vectors which transduce dividing cells (e.g., vectors derived from murine leukemia virus; see, e.g., Miller and Baltimore, Mol. Cell. Biol. 6:2895 (1986)) can be used. The production of a recombinant retroviral vector carrying a gene of interest is typically achieved in two stages. First, a Rheb gene can be inserted into a retroviral vector which contains the sequences necessary for the efficient expression of the Rheb gene (including promoter and/or enhancer elements which can be provided by the viral long terminal repeats (LTRs) or by an internal promoter/enhancer and relevant splicing signals), sequences required for the efficient packaging of the viral RNA into infectious virions (e.g., a packaging signal (Psi), a tRNA primer binding site (−PBS), a 3′ regulatory sequence required for reverse transcription (+PBS)), and a viral LTRs). The LTRs contain sequences required for the association of viral genomic RNA, reverse transcriptase and integrase functions, and sequences involved in directing the expression of the genomic RNA to be packaged in viral particles.

Following the construction of the recombinant vector, the vector DNA is introduced into a packaging cell line. Packaging cell lines provide viral proteins required in trans for the packaging of viral genomic RNA into viral particles having the desired host range (i.e., the viral-encoded core (gag), polymerase (pol) and envelope (env) proteins). The host range is controlled, in part, by the type of envelope gene product expressed on the surface of the viral particle. Packaging cell lines can express ecotrophic, amphotropic or xenotropic envelope gene products. Alternatively, the packaging cell line can lack sequences encoding a viral envelope (env) protein. In this case, the packaging cell line can package the viral genome into particles which lack a membrane-associated protein (e.g., an env protein). To produce viral particles containing a membrane-associated protein which permit entry of the virus into a cell, the packaging cell line containing the retroviral sequences can be transfected with sequences encoding a membrane-associated protein (e.g., the G protein of vesicular stomatitis virus (VSV)). The transfected packaging cell can then produce viral particles which contain the membrane-associated protein expressed by the transfected packaging cell line; these viral particles that contain viral genomic RNA derived from one virus encapsidated by the envelope proteins of another virus are said to be pseudotyped virus particles.

Oocytes which have not undergone the final stages of gametogenesis are typically infected with the retroviral vector (e.g., such as by injection of viral DNA or particles). The infected oocytes are then permitted to complete maturation with the accompanying meiotic divisions. The breakdown of the nuclear envelope during meiosis permits the integration of the proviral form of the retrovirus vector into the genome of the oocyte. When pre-maturation oocytes are used, the infected oocytes are then cultured in vitro under conditions that permit maturation of the oocyte prior to fertilization in vitro. Conditions for the maturation of oocytes from a number of mammalian species (e.g., bovine, ovine, porcine, murine, and caprine) are well known in the art. In general, a base medium for in vitro maturation of bovine oocytes can be used (e.g., TC-M199 medium supplemented with hormones (e.g., luteinizing hormone and estradiol)). Other media for the maturation of oocytes can be used for the in vitro maturation of other mammalian oocytes and are well known to the skilled artisan. The amount of time a pre-maturation oocyte is exposed to maturation medium to permit maturation varies between mammalian species, as is known to the skilled artisan. For example, an exposure of about 24 hours is sufficient to permit maturation of bovine oocytes, while porcine oocytes require about 44-48 hours.

Oocytes can be matured in vivo and employed in place of oocytes matured in vitro. For example, when porcine oocytes are employed, matured pre-fertilization oocytes can be harvested directly from pigs that are induced to superovulate. Briefly, on day 15 or 16 of estrus, a female pig(s) can be injected with about 1000 units of pregnant mare's serum (PMS; available from Sigma and Calbiochem). Approximately 48 hours later, the pig(s) is injected with about 1000 units of human chorionic gonadotropin) (hCG; Sigma), and 24-48 hours later matured oocytes are collected from oviduct. These in vivo matured pre-fertilization oocytes can then be injected with the desired preparation. Methods for the superovulation and collection of in vivo matured (e.g., oocytes at the metaphase 2 stage) oocytes are known for a variety of mammals (e.g., for superovulation of mice, see Hogan et al., in Manipulating the Mouse Embryo: A Laboratory Manual, 2nd ed., Cold Spring Harbor Laboratory Press, Cold Spring Harbor, N.Y. (1994), pp. 130-133; the disclosure of which is incorporated by reference herein in its entirety).

Retroviral vectors capable of infecting the desired species of non-human animal can be grown and concentrated to very high titers (e.g., 1×10⁸ cfu/ml). The use of high titer virus stocks allows the introduction of a defined number of viral particles into the perivitelline space of each injected oocyte. The perivitelline space of most mammalian oocytes can accommodate about 10 picoliters of injected fluid (those skilled in the art know that the volume that can be injected into the perivitelline space of a mammalian oocyte or zygote varies somewhat between species as the volume of an oocyte is smaller than that of a zygote and thus, oocytes can accommodate somewhat less than can zygotes). The virus stock can be titered and diluted prior to microinjection into the perivitelline space so that the number of proviruses integrated in the resulting transgenic animal is controlled. The use of pre-maturation oocytes or mature fertilized oocytes as the recipient of the virus minimizes the production of animals which are mosaic for the provirus as the virus integrates into the genome of the oocyte prior to the occurrence of cell cleavage.

Prior to microinjection of the titered and diluted (if required) virus stock, the cumulus cell layer can be opened to provide access to the perivitelline space. The cumulus cell layer need not be completely removed from the oocyte and indeed for certain species of animals (e.g., cows, sheep, pigs, or mice), a portion of the cumulus cell layer remains in contact with the oocyte to permit proper development and fertilization post-injection. Injection of viral particles into the perivitelline space allows the vector RNA (i.e., the viral genome) to enter the cell through the plasma membrane thereby allowing proper reverse transcription of the viral RNA. The presence of the retroviral genome in cells (e.g., oocytes or embryos) infected with pseudotyped retrovirus can be detected using a variety of means, such as those described herein or as otherwise known to the skilled artisan.

In an exemplary embodiment, the Rheb gene can be introduced into avian species using a viral vector as described in U.S. Pat. No. 5,162,215 (the disclosure of which is incorporated by reference herein in its entirety). Alternatively, a Rheb gene expression vector or transfected cells producing the expression vector (e.g., a virus containing the Rheb gene) is injected into developing avian oocytes in vivo, for example, as described in Shuman and Shoffner (Poultry Science 65:1437-44 (1986), which is incorporated by reference herein in its entirety).

The overall efficiency of the nucleic acid delivery procedure to avian cells can depend on the methods and timing of gene delivery. Infection efficiency is optionally increased by, for example, subjecting the blastoderm or cells derived from the blastoderm to several rounds of infection or adding a selectable marker (e.g., an antibiotic resistance gene) in combination with the Rheb gene and infusing the antibiotic into the yolk or testes following transfection or cell transfer.

In another embodiment, a transgenic animal is prepared by nuclear transfer. The terms “nuclear transfer” or “nuclear transplantation” refer to methods of preparing transgenic animals wherein the nucleus from a donor cell is transplanted into an enucleated oocyte. Nuclear transfer techniques or nuclear transplantation techniques are known in the art. (See, e.g., Campbell et al., Theriogenology 43:181 (1995); Collas and Barnes, Mol. Reprod. Dev. 38:264-67 (1994); Keefer et al., Biol. Reprod. 50:935-39 (1994); Sims et al., Proc. Natl. Acad. Sci. USA 90:6143-47 (1993); Prather et al., Biol. Reprod. 37:59-86 (1988); Roble et al., J Anim. Sci. 64:642-64 (1987); International Patent Publications WO 90/03432, WO 94/24274, and WO 94/26884; U.S. Pat. Nos. 4,994,384 and 5,057,420; the disclosures of which are incorporated by reference herein in their entirety.) For example, nuclei of transgenic embryos, pluripotent cells, totipotent cells, embryonic stem cells, germ cells, fetal cells or adult cells can be transplanted into enucleated oocytes, each of which is thereafter cultured to the blastocyst stage. (As used herein, the term “enucleated” refers to cells from which the nucleus has been removed as well as to cells in which the nucleus has been rendered functionally inactive.) The nucleus containing a Rheb gene can be introduced into these cells by any method known to the skilled artisan, including those described herein. The transgenic cell is then typically cultured in vitro to the form a pre-implantation embryo, which can be implanted in a suitable female (e.g., a pseudo-pregnant female).

The transgenic embryos optionally can be subjected, or resubjected, to another round of nuclear transplantation. Additional rounds of nuclear transplantation cloning can be useful when the original transferred nucleus is from an adult cell (i.e., fibroblasts or other highly or terminally differentiated cell) to produce healthy transgenic animals.

Other methods for producing a Rheb transgenic animal include methods adapted to use male sperm cells to carry the Rheb gene to an egg. In one example, a Rheb gene can be administered to a male animal's testis in vivo by direct delivery. The Rheb gene can be introduced into the seminiferous tubules, into the rete testis, into the vas efferens or vasa efferentia using, for example, a micropipette. To ensure a steady infusion of the gene delivery mixture, the injection can be made through the micropipette with the aid of a picopump delivering a precise measured volume under controlled amounts of pressure.

Alternatively, the Rheb gene can be introduced ex vivo into the genome of male germ cells. A number of known gene delivery methods can be used for the uptake of nucleic acid sequences into the cell. Suitable methods for introducing Rheb genes into male germ cells include, for example, liposomes, retroviral vectors, adenoviral vectors, adenovirus-enhanced gene delivery systems, or combinations thereof. Whether introduced in vivo or in vitro, the Rheb gene, once in contact with the male germ cells, is taken up and transported into the appropriate cell location for integration into the genome and expression.

Following transfer of a Rheb gene to male germ cells by any suitable method, a transgenic zygote can be formed by breeding the male animal with a female animal. The transgenic zygote can be formed, for example, by natural mating (e.g., copulation by the male and female vertebrates of the same species), or by in vitro or in vivo artificial means. Suitable artificial means include, but are not limited to, artificial insemination, in vitro fertilization (IVF) and/or other artificial reproductive technologies, such as intracytoplasmic sperm injection (ICSI), subzonal insemination (SUZI), partial zona dissection (PZD), and the like, as will be appreciated by the skilled artisan. (See, e.g., International Patent Publication WO 00/09674, the disclosure of which is incorporated by reference herein in its entirety.)

In yet another aspect, methods are provided to identify subjects in need of Rheb agonist or Rheb antagonist therapy. Such methods are typically performed by detecting changes in Rheb activity, as compared with control cells. Suitable assays to detect changes in Rheb activity in in vitro, ex vivo and in vivo assays can further include, for example, monitoring Rheb protein and/or message levels. Rheb is a dose-dependent effector. Levels of Rheb protein or RNA can be measured relative to control cells to determine whether a subject exhibits a change Rheb activity. For example, Rheb protein levels can be measured by immunoassay using antibody against Rheb protein. Suitable immunoassays include, for example, competitive and non-competitive assay systems using techniques such as radioimmunoassays, ELISA (enzyme linked immunosorbent assay) “sandwich” immunoassays, immunoradiometric assays, in situ immunoassays (using colloidal gold, enzyme or radioisotope labels, and the like), Western blots, immunofluorescence assays, protein A assays, immunoelectrophoresis assays, and the like. (See generally Harlow and Lane, 1999 (supra); Harlow and Lane, 1988 (supra).) Similarly, Rheb RNA levels can be measured by suitable assay, such as for example, polymerase chain reaction assay, Southern blotting, Northern blotting, or the like. (See generally Sambrook (supra); Ausubel et al. (supra). In addition, assays can be used to detect Rheb gene amplification. Suitable assays include for example, Southern blotting, polymerase chain reaction, and the like. (See generally Sambrook et al. (supra); Ausubel et al. (supra).) In addition, Rheb activity can be measured by Rheb-GTP/Rheb-GDP ratio, where Rheb-GTP is the active form. Such assays are described, for example, in Zhang et al. (Nat. Cell Biol. 5:578-81 (2003); the disclosure of which is incorporated by reference herein). Because Rheb-GTP levels are responsive to insulin, changes in upstream signaling can also be determined.

The following examples are provided merely as illustrative of various aspects of the invention and shall not be construed to limit the invention in any way.

### EXAMPLES

The following studies demonstrate that Rheb has a nutrient-sensing function and functions as a regulator of cellular growth.

Materials and Methods

Flystocks and transgenes: P[GS1093] was mobilized using D2-3 transposase (Robertson et al., Genetics 118:461-70 (1988)) and the location of GSjE2 mapped to the first exon of rheb using RT-PCR. (Toba et al., Genetics 151:725-37 (1999).) Rheb^{PΔ1} and rheb^{PΔ2} were created by mobilization of GSjE2 using Δ2-3 transposase (Robertson et al., Genetics 118:461-70 (1988)), and deletions mapped using PCR with a series of primers to neighboring genes as well as sequencing PCR products spanning the deletions using Big Dye 3.0 (PE-Biosystems) and an Applied Biosystems 377 Sequencer. The primers used to amplify and sequence across the deletion of rhebPΔ1 were as follows: 5′-ACGGGCCTTG ATATTTTCTG-3′ (SEQ ID NO:1) and 5′-GCACAAGTTCGCTG TTTGAA-3′ (SEQ ID NO:2). The primers used to amplify and sequence across the deletion of rhebPΔ2 were as follows: 5′-GTGGCAGTACCCT GGAAAAA-3′ (SEQ ID NO:3) and 5′-CAAGACAACCGCTCT TCTCC-3′ (SEQ ID NO:4). To make the UAS-Rheb transgene, a full-length EST of Rheb (GH10361, Research Genetics) was digested with Xho I/Bgl II, cloned into pUAST (Brand and Perrimon, Genes Dev. 8:629-39 (1994)) and transformed into w; +; +flies.

Other flystocks used in these studies were as follows:
w; gmrGAL4/Cyo; +(Freeman, Cell 87:651-60. (1996))
w; enGAL4; +(Brand and Perrimon, Genes Dev. 8:629-39 (1994))
ywhsflp¹²²; +;Act>cd2>GAL4, UASGFP (Pignoni and Zipursky, Development 124:271-78 (1997); Neufeld et al., Cell 93:1183-93 (1998))
w; tGPH; act>cd2>GAL4/tm6b (Britton et al., Dev. Cell 2:239-49 (2002)
w; UASPTEN; +(Gao et al., Dev. Biol. 221:404-18. (2000)
w; UASTSC1,UASTSC2/CyO; Sb/Tm6 (Potter et al., Cell 105:357-68 (2001))
ywhsflp¹²²; dTOR^{ΔP}FRT40A/Sm6Tm6 (Zhang et al., Genes Dev. 14:2712-24 (2000))
w; +; dS6¹⁻¹/Tm6b (Montagne et al., Science 285:2126-29 (1999)
hsflp¹²²; FRT40AtubGAL80; tubGAL4/Tm6b (Lee and Luo, Neuron 22:451-61 (1999))
w; hsGAL4/CyO; +(Bloomington Stock #2077)
hsflp¹²²; hs[neo]FRT40A; +(Bloomington stock #1802)

Scanning electron microscopy: Female flies were fixed and dehydrated in ethanol then immersed overnight in pure hexamethyldisilazane before mounting and sputter coating with 30 nm of gold-palladium. Electron microscopy was performed using a JEOL JSM5800 scanning electron microscope. All images were taken at 90-fold magnification.

Northern analyses: First instar larva homozygous for deletion of rheb were sorted apart from heterozygote siblings containing a GFP-marked balancer chromosome. Total RNA was isolated using TRIzol Reagent (Invitrogen) and 5 μg loaded onto a standard 1.2% agarose gel containing 2% formaldehyde. RNA was transferred, probed, and detected according to the manufacturer's protocol (DIG Northern Starter Kit, Roche). In vitro transcribed DIG-labeled probes were generated using cDNAs for Rheb (GH10361, Research Genetics) and Rp49 (O'Connell and Rosbash, Nucleic Acids Res. 12:5495-13 (1984)).

Clonal analyses: Random GAL4-expressing clones in fat body tissue resulting from heat shock independent events (Britton et al., Dev. Cell 2:239-49 (2002)) were examined in wandering, fed L3 larvae or protein-starved L2 larvae (raised on 20% sucrose in PBS) following fixation in 4% paraformaldehyde, staining with Hoechst 33258, and dissection. DNA intensity and cell size was measured using histogram functions of Adobe Photoshop. Random clones in wing discs were generated in animals raised at 25° C. by heat shocking at 37° C. for 20 minutes at 72 hours AED and fixing as above at 120 hours AED. Wing discs were stained with Hoechst 33258, mounted, and the number of cells/clone enumerated using a Leica DMRB Microscope. Cell doubling times were calculated as (log2/logN)hr, with N as the mean number cells/clone and hr as the time between heat shock and fixation.

Flow cytometry: In studies inducing expression of Rheb, random clones were generated in animals raised at room temperature by heat shocking at 37° C. for one hour at 88 hours AED. In studies where Rheb was induced in the presence or absence of tor using the GAL4/GAL80 system (Lee and Luo, Neuron 22:451-61 (1999)), FLP/FRT recombination was induced in animals raised at room temperature by heat shocking for 1 hour 30 minutes at 36 and 60 hours AED. The animals were dissected at wandering and flow cytometry performed on dissociated wing imaginal discs as previously described (Neufeld et al., Cell 93:1183-93 (1998)).

Characterization of Rheb in S2 cells: A full-length Rheb cDNA was myc-tagged at the N-terminus and cloned in the pAc5.1/V5-HisB vector (Invitrogen) as described previously (Gao and Pan, Genes Dev. 15:1383-92 (2001)). HA-S6K expression construct has been described previously (Zhang et al., Genes Dev. 14:2712-24 (2000)). Drosophila cell culture, transfection, RNAi and western blotting were carried according to standard procedures (Gao et al., Nat. Cell Biol. 4:699-704 (2002)). Mammalian CYP7A1 was used as control for an RNAi study (Gao et al., supra). Antibodies against myc, HA and Phospho T-398-S6K were from Santa Cruz Biotechnology, Sigma and Cell Signaling Technology, respectively. Antibody against TSC2 was a gift from Naoto Ito.

Microarray analyses: For each hybridization, total RNA was isolated from approximately 50 larvae in the second instar using TRIzol Reagent (Invitrogen) followed by RNeasy (Qiagen) clean up. Expression profiles were performed using spotted microarrays constructed from release 1 of the Drosophila Gene Collection and 430 additional sequences. Target label preparation and hybridization protocols were performed according to publicly available protocols. (See, e.g., the web site for the Fred Hutchinson Cancer Research Center, under Shared Resources in the protocols for genomics.) Spot intensities were filtered and removed if the values did not exceed 250 units above background or if a spot was flagged as questionable by the GenePix Pro software. Spot level intensity was log₂ transformed and centralized applied using Microsoft Excel to correct for intra-array intensity-dependent ratio biasing. Each study was replicated 5 times (including reversal of dye orientation). Significance Analysis of Microarrays (SAM) (Tusher et al., Proc. Natl. Acad. Sci. USA 98:5116-21 (2001)) was used to select statistically significant data and a two-class paired test was conducted using a 1.7-fold threshold and a false detection rate of <5%.

Results

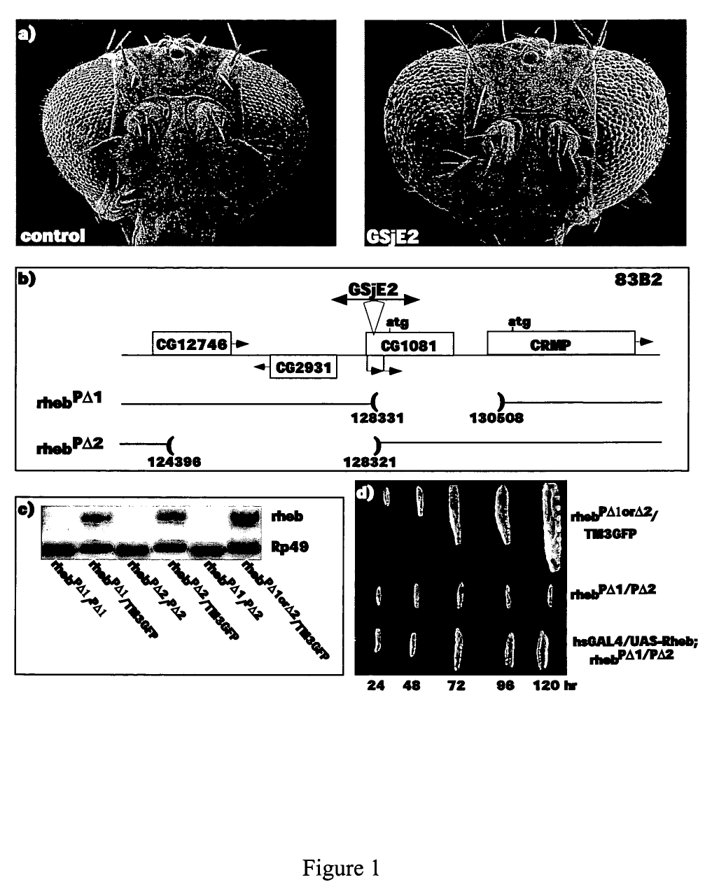

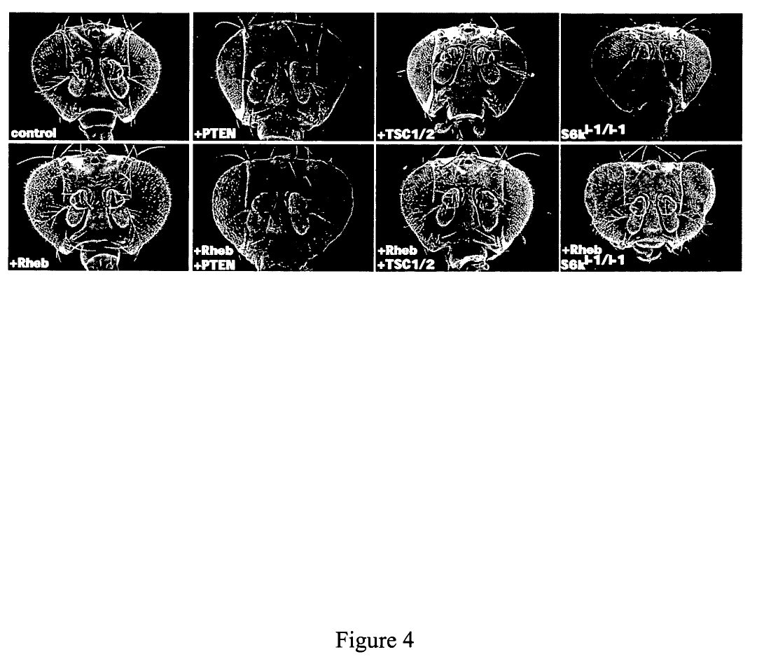

Identification of Rheb as a promoter of growth: A gain-of-function screen utilizing the GeneSearch (GS) P-element was employed to identify novel regulators of cell growth. Transcription from mobilized P-elements was induced using gmrGAL4, which is expressed in post-mitotic cells of the developing eye (Ellis et al., Development 119:855-65 (1993)). Of approximately 20,000 animals scored, 48 were found to have enlarged eyes and were therefore established as lines. One line, which demonstrated one of the strongest overgrowth phenotypes, was GSjE2 (FIG. 1a). The flanking sequences of GSjE2 were identified using RT-PCR (Toba et al., Genetics 151:725-37 (1999)) and indicated that the P element was located at cytological map position 83B2, within the 5'UTR of CG1081 (FIG. 1b). Sequence alignments indicated that CG1081 was the Drosophila homologue of the gene, rheb, a member of the Ras superfamily of GTP-binding proteins. Similar to the previously described mammalian and yeast homologues, Drosophila Rheb encodes a carboxy-terminal CAAX farnesylation motif and contains arginine and serine residues at positions 15 and 16. To verify that over-expression of Rheb was responsible for the phenotype, a full-length EST (GH10361) was cloned downstream of UAS sequences and transformed into 5 naïve flies. Multiple independently derived transgenic animals demonstrated a recapitulation of the eye phenotype (FIG. 4), confirming that induction of Rheb alone was sufficient for the overgrowth seen in the original GSjE2 line.

Rheb is required for larval development: Imprecise excision of the GS element in the 5′ UTR of rheb yielded two lines which showed no detectable mRNA for rheb (FIG. 1c). PCR and sequencing of genomic DNA revealed that one allele, rheb^{PΔ1}, removed all of the coding sequence for rheb and 13 bases of the 5'UTR transcript of the neighboring gene, Collapsin Response Mediator Protein (CRMP) (FIG. 1b). Northern analyses showed this line still expresses CRMP. Additionally, transheterozygote animals containing the rheb^{PΔ1} allele and a recessive lethal located within CRMP (Bloomington stock #14252) were viable, suggesting that rheb^{PΔ1} adequately expresses CRMP. The second line, rheb^{PΔ2}, deleted sequences in the opposing direction, removing the promoter of rheb as well as coding sequence for two predicted genes located upstream of rheb (FIG. 1b). Animals homozygous for either excision survive throughout embryogenesis, though this may be due to maternal contribution of Rheb message that was detected using in situ hybridization. However, the mutant animals spend an extended period in the first instar of larval development before dying approximately 6 days after hatching. Additionally, transheterozygotes containing these two opposing deletions show the same L1 growth arrest phenotype (FIG. 1d). Because these rheb^{PΔ1/PΔ2} animals are only homozygous for disruption of rheb, it is likely that loss of rheb is responsible for lethality. To support this interpretation, UAS-Rheb and hsGAL4 were introduced into the transheterozygous rheb^{PΔ1/PΔ2} animals. With or without heat-shock, addition of these transgenes partially rescued the growth phenotype, allowing the rheb^{PΔ1/PΔ2} animals to reach the second larval stage before arresting (FIG. 1d). The inability to fully rescue the rheb^{PΔ1/PΔ2} animals is perhaps due to inadequately reproducing the expression of endogenous Rheb. No obvious reason for lethality of rheb^{PΔ1/PΔ2} animals was apparent. Food was detected in the gut of mutant animals, verifying that they were eating. This result suggests that inhibition of larval development may be due to a cellular growth defect.

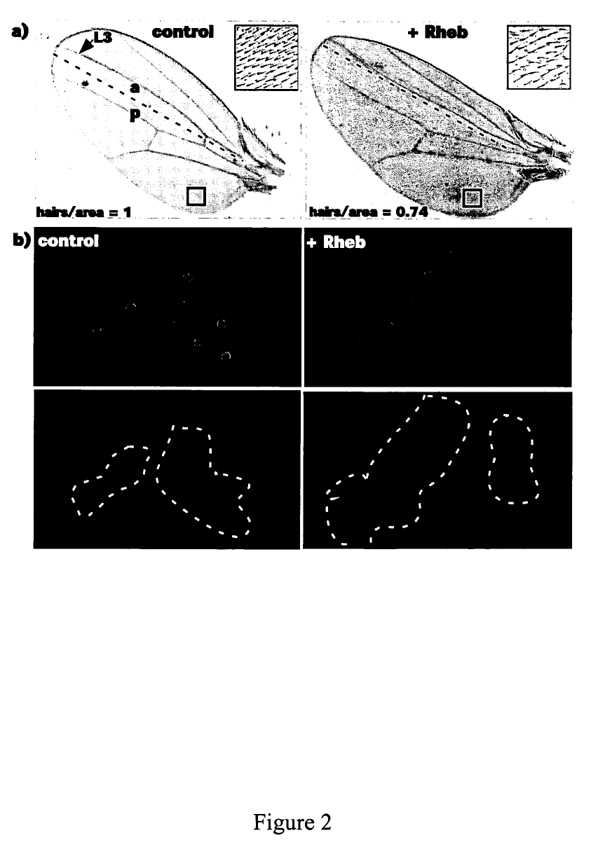

Over-expression of Rheb increases cell size in multiple tissues: To ascertain whether Rheb functions as a general promoter of growth, the effect of Rheb over-expression was examined in multiple tissues. Expression of Rheb in the posterior compartment of the wing using the enGAL4 driver resulted in an expansion of the posterior half of the adult wing with minimal disruption of patterning or cell fate (FIG. 2a). Measurement of the area between the L3 vein and posterior margin revealed that expression of Rheb resulted in an 11% increase in tissue mass. It was evident that the wing hairs (trichomes) of the posterior wing were spaced further apart than controls (FIG. 2a). Because a single hair marks each wing cell, the total hair number within a defined area was enumerated as a means of gauging cell size. EnGAL4, UAS-Rheb animals had only 74% the cell density of controls in posterior compartments, indicating that over-expression of Rheb leads to cell enlargement in the adult wing. To examine the effect of Rheb in larval tissues, random clones of cells over-expressing Rheb and GFP were generated using the flip/GAL4 method (Struhl and Basler, Cell 72:527-40 (1993); Pignoni and Zipursky, Development 124:271-78. (1997); Neufeld et al., Cell 93:1183-93 (1998)). Rheb expression resulted in increased cell size and nuclear DNA content in endoreduplicating tissues including the gut, proventriculus, and fat body. Fat body cells over-expressing Rheb encompassed about 2.5 times the area of control cells and contained, on average, 64% more DNA as determined by staining with Hoechst (FIG. 2b). These data indicate that Rheb promotes growth in both mitotic and endoreduplicating cells of various tissues.

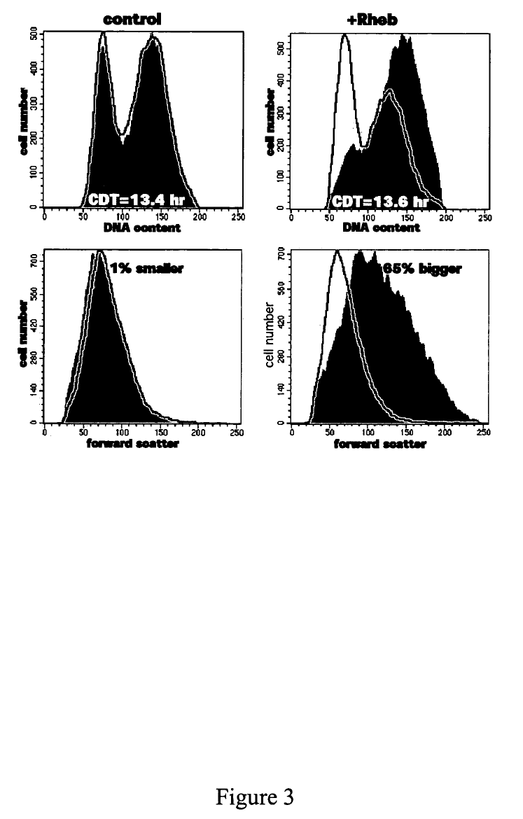

Rheb promotes G1/S progression but does not accelerate cell division: The above studies demonstrate that Rheb functions to promote cell growth. To determine if this increased growth was accompanied by accelerated cell cycle progression, clones of cells over-expressing Rheb generated in developing wing discs were examined using the flip/GAL4 method (Struhl and Basler, Cell 72:527-40 (1993); Pignoni and Zipursky, Development 124:271-78 (1997); Neufeld et al., Cell 93:1183-93 (1998)). Cell cycle profiles were obtained by performing flow cytometry on live cells following dissociation of wing discs (FIG. 3). Forward scatter (FSC) analysis was used as an approximation of cell volume and confirmed Rheb's effect on cell size—demonstrating a 65% increase in mean FSC in the transgenic line with the strongest phenotype. DNA profiles revealed that over-expression of Rheb leads to a profound decrease in the population of cells with a G1 content of DNA (approximately 75% fewer cells than control, FIG. 3). Next, cell division times were calculated by counting the number of cells per clone and monitoring the time between clone induction and fixation of the wing disc (Neufeld et al., Cell 93:1183-93 (1998)). The doubling time of control cells and cells over-expressing Rheb was calculated to be 13.4 hours (N=236 clones) and 13.6 hours (N=366 clones), respectively. These results indicate that although over-expression of Rheb strongly promotes G1/S progression, there must be a corresponding extension of the time spent in G2/M that results in the overall preservation of a normal rate of cell division.

Rheb interacts with components of the insulin/PI3K and TOR signaling pathways: The growth and cell cycle phenotypes caused by Rheb are reminiscent of those caused by hyperactivation of insulin/PI3 kinase (PI3K) signaling (Weinkove and Leevers, Curr. Opin. Genet. Dev. 10:75-80 (2000); Potter and Xu, Curr. Opin. Genet. Dev. 11:279-86 (2001)). Using a PH-GFP reporter of PI3K activity(Britton et al., Dev. Cell. 2:239-49 (2002)), it was found that Rheb did not stimulate P13K function, suggesting that if Rheb has a role in insulin/PI3K signaling, it must act further downstream.

Genetic interactions of rheb with components that negatively regulate the output of insulin/PI3K activity were analyzed. PTEN directly antagonizes the kinase function of P13K and suppresses growth when overexpressed (Goberdhan et al., Genes Dev. 13:3244-58 (1999); Huang et al., Development 126:5365-72 (1999); Gao et al., Dev. Biol. 221:404-18 (2000)). Co-over-expression of Rheb bypassed PTEN-mediated growth inhibition in the adult eye (FIG. 4), confirming that Rheb functions downstream of P13K activity. Tuberous sclerosis complex 1 and 2 (TSC 1/2) is a phosphorylation target of PKB and has recently been demonstrated to interfere with insulin/PI3K signaling (Inoki et al., Nat. Cell. Biol. 4:648-57 (2002); Potter et al., Nat. Cell. Biol. 4:658-65 (2002); Manning et al., Mol. Cell 10:151-62 (2002); Tapon et al., Cell 105:345-55 (2001); Potter et al., Cell 105:357-68 (2001); Gao and Pan, Genes Dev. 15:1383-92 (2001)).

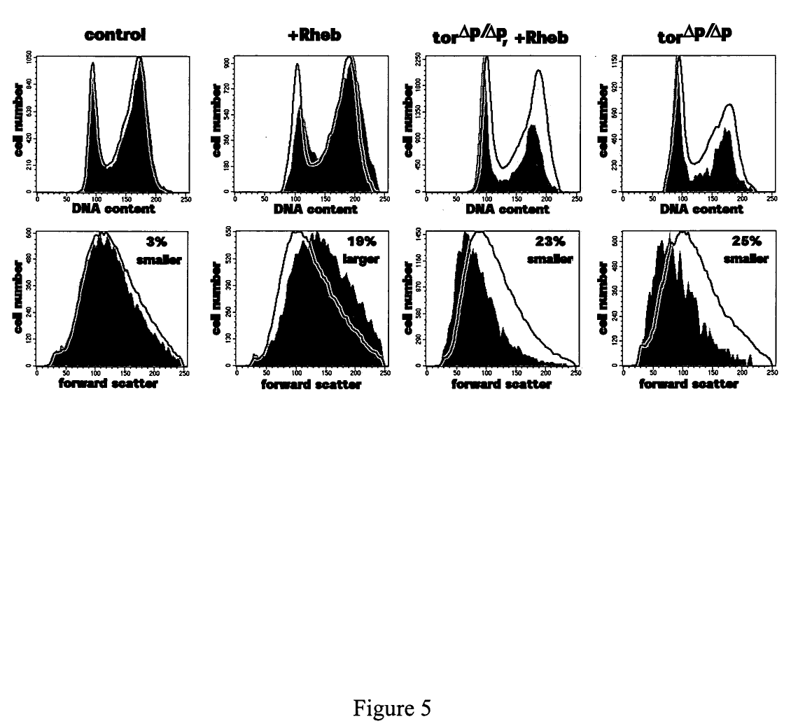

Over-expression of TSC1/2 greatly reduced the size of the adult eye, and this growth suppression was partially overcome by co-expression of Rheb (FIG. 4). The TSC1/2 complex likely antagonizes growth by suppressing the target of rapamycin (TOR), a protein implicated in mediating protein synthesis in response to nutrients (reviewed in Schmelzle and Hall, Cell 103:253-62 (2000)). TSC1/2 and TOR physically associate (Gao et al., Nat. Cell. Biol. 4:699-704 (2002)) and over-expression of TSC1/2 inhibits TOR signaling (Inoki et al., supra; Gao et al., supra). Genetic epistasis tests place TOR downstream of TSC1/2 (Gao et al., supra.) In addition, TOR has been shown to be necessary for insulin/PI3K-directed growth (Zhang et al., Genes Dev. 14:2712-24 (2000)). The ability of Rheb to induce cell growth was tested in the absence of tor. Clones of cells that lacked tor were created in developing wing discs using FRT-mediated recombination and were examined using flow cytometry in the absence or presence of overexpressed Rheb (FIG. 5; Lee and Luo, Neuron 22:451-61 (1999)). As previously described (Zhang et al., Genes Dev. 14:2712-24 (2000); Oldham et al., Genes Dev. 14:2689-94 (2000)), loss of tor leads to a marked reduction in cell size and a decrease in the population of cells in the S and G2 phases of the cell cycle. This phenotype persisted when Rheb was overexpressed, confirming that TOR is epistatic to overexpressed Rheb.

In addition, the role of S6 kinase (S6K), a protein involved in translation and an effector of TOR-mediated growth, was examined. In animals null for s6k, Rheb was still able to produce enlarged eyes when expressed using gmrGAL4 (FIG. 4). The puckering of eye tissue in s6k animals over-expressing Rheb is likely due to the reduced body and head capsule size of s6k animals (see, e.g., Montagne et al., Science 285:2126-29 (1999)). Radimerski et al. similarly reported that P13K over-expression still promoted growth in animals lacking S6K (Radimerski et al., Nat. Cell Biol. 4:251-55. (2002)). In conclusion, these genetic interaction tests indicate that Rheb induces cell growth either as a downstream component of insulin/PI3K signaling or in a parallel pathway that requires TOR.

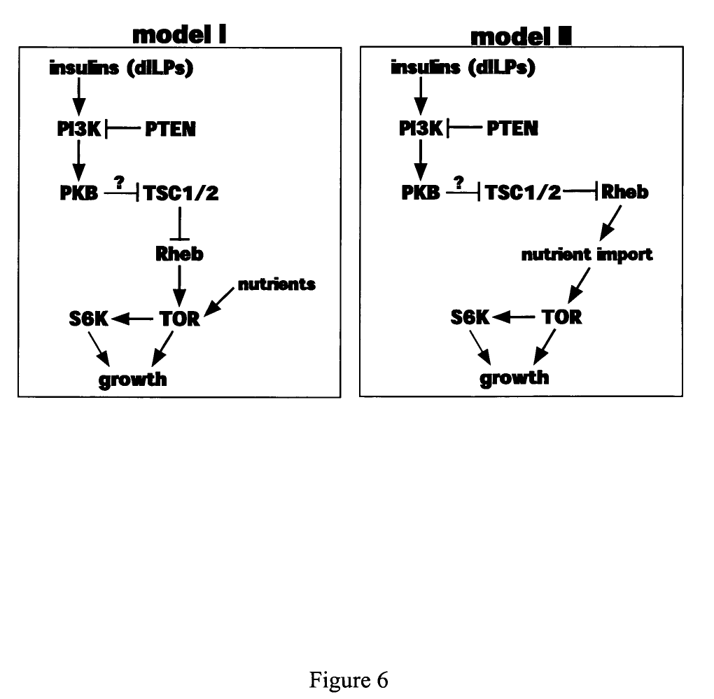

Rheb regulates TOR/S6K signaling in Drosophila cells. To further dissect how Rheb interfaces with TOR, a biochemical readout of TOR function, S6K activity, was used. Tagged S6K and/or Rheb constructs were transfected into Drosophila S2 cells, immunoprecipitated from cell lysates, and activation of S6K activity was measured using a phospho-specific antibody (Radimerski et al., supra). Over-expression of Rheb led to an increase of activated S6K (FIG. 6a). Although S6K is normally inactivated in response to amino acid starvation, Rheb-mediated activation of S6K persisted in the absence of amino acids (FIG. 6b). Recently, loss of TSC1 or TSC2 was demonstrated to lead to a similar increase in S6K activity which is also resistant to amino acid withdrawal (Gao et al. (2002), supra.) RNA interference was used to examine the relationship between TSC2 and Rheb in modulation of S6K function. Whereas loss of TSC2 resulted in a persistence of S6K activity in media free of amino acids, loss of Rheb abolished S6K activity regardless of the presence of amino acids (FIG. 6c). In the absence of both TSC2 and Rheb, S6K remained inactive, indicating that Rheb is epistatic to TSC2 and that Rheb is required for S6K activity.

Rheb and nutrition: To ascertain when and where Rheb is normally utilized to regulate growth, in situ hybridization to mRNA was performed. This analysis revealed that rheb is expressed ubiquitously throughout embryogenesis and in both mitotic and endoreduplicative tissues of L3 larva. Next, the nutritional responsiveness of rheb expression was examined. Microarray analyses revealed that rheb transcripts were upregulated in larvae that were starved on a protein-free diet. The induction of rheb was rapid (2.2-fold at 4 hours, p=0.0009) and persistent (2.4-fold at 48 hours, p=0.001). Upon refeeding, levels of rheb decreased 2-fold (p=0.0005). Because microarray analyses were performed on whole animals, in situ hybridization to RNA was used to examine whether rheb expression was induced in a tissue-specific manner in response to protein starvation. No patterned increase in rheb levels was apparent, suggesting that rheb was uniformly induced throughout the animals.

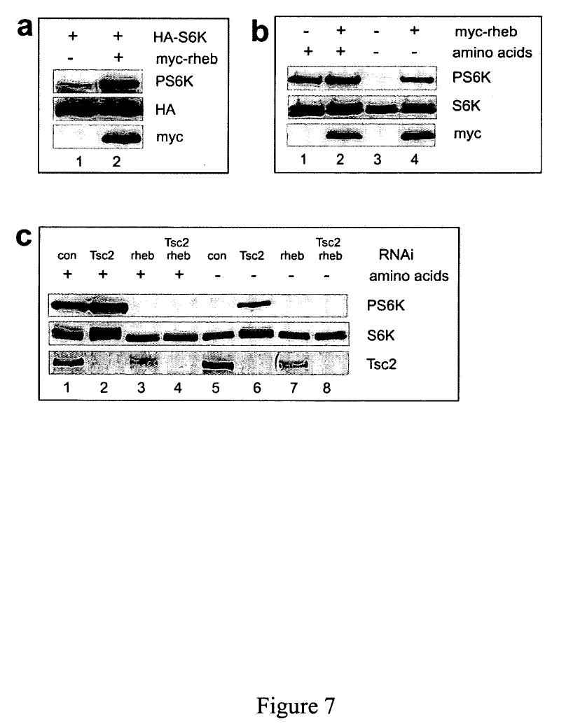

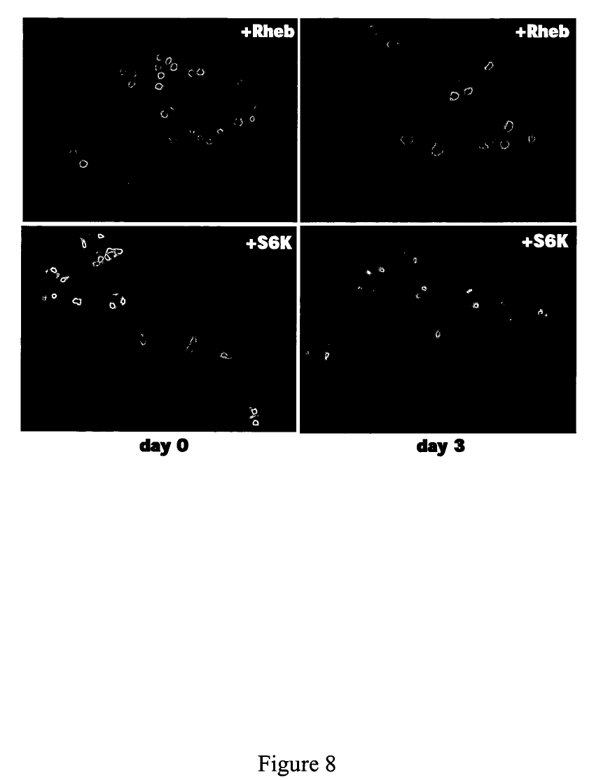

To investigate whether Rheb still functioned as a growth promoter in starved animals, cells that over-expressed Rheb were produced in the fat body of young larvae that were starved for 72 hours. Prior to starvation, fat body cells expressing Rheb were approximately the same size as control cells in the same tissue (FIG. 7). Following three days of starvation, no growth of control cells was apparent but cells over-expressing Rheb demonstrated impressive growth. Thus, Rheb is capable of bypassing the nutritional requirement for growth. Constitutive expression of S6K in starved animals failed to promote cell growth (FIG. 7) indicating that S6K alone cannot recapitulate the phenotype observed with Rheb.

Discussion

This study of Drosophila Rheb has revealed a new function for this small GTP-binding protein in regulating cell growth. Loss of rheb suspends larval growth while over-expression of Rheb leads to autonomous increases in cell size and acceleration through G1/S. Interestingly, Rheb did not accelerate the cell division cycle in mitotic cells and was incapable of promoting unscheduled proliferation in post-mitotic cells of the pupal eye. In comparison to similar studies on activated Ras (Ras1^{V12}) in Drosophila (Prober and Edgar, Cell 100:435-46 (2000); Prober and Edgar, Genes Dev. 16:2286-2299 (2002)), Rheb is a far more potent promoter of growth but effects none of the correspondent alterations of cell fate caused by Ras 1^{V12} over-expression in the wing and eye. The patterning phenotypes resulting from expressing Ras1^{V12} in the eye dominated in co-expression studies with Rheb, suggesting that Rheb does not antagonize cell fate determination by Ras1^{V12}. Because Raf-1 is an effector of Ras signaling in directing cell fate in Drosophila (Dickson et al., Nature 360:600-03 (1992)), these results suggest that Rheb does not affect Raf-1 function in vivo as predicted by in vitro binding studies (Yee and Worley, supra; Clark et al., supra).

Rheb over-expression phenotypes most closely resemble those caused by hyperactivation of insulin/PI3K signaling (Weinkove and Leevers, Curr. Opin. Genet. Dev. 10:75-80 (2000); Potter and Xu, Curr. Opin. Genet. Dev. 11:279-86 (2001)). Rheb-induced overgrowth was able to bypass two negative regulators in this pathway, PTEN and TSC1/2, suggesting that Rheb acts further downstream. RNA interference studies in cultured cells demonstrated that Rheb is epistatic to TSC1/2. Interestingly, TSC2 contains a GTPase-activating domain (GAP). Although it was initially predicted that Rheb is not regulated by GTP/GDP exchange (reviewed in Reuther and Der, Curr. Opin. Cell. Bio. 12:157-65 (2000)), these predictions are primarily based on activating mutations in Ras. The recent results of Im et al. (supra), demonstrating that the high activation state of Rheb was not due to the corresponding amino acid substitutions of oncogenic Ras (amino acids 15 and 16) indicate that Ras and Rheb may be regulated differently by GAPs/GEFs. Either Rheb GEFs are in great excess or the activity of Rheb GAPs is insensitive to amino acids alterations at positions 15 and 16.

Inactivation of TSC1 or TSC2 results in tumorigenesis in humans (reviewed in Young and Povey, Mol. Med. Today 4:313-19 (1998)) and mutations in the GAP domain of TSC2 have been identified in patients (Maheshwar et al., Hum. Mol. Genet. 6:1991-96 (1997)). If Rheb is a physiological target of TSC2, a greater proportion of Rheb should be GTP-bound in these patients. Alternatively, rather than serving to augment GTPase activity towards Rheb, TSC1/2 may antagonize Rheb physically. TSC1/2 has been reported to be located at the cell membrane and this localization is disrupted by PKB signaling (Potter et al., Nat. Cell. Biol. 4:658-65 (2002)). Rheb has been shown to be farnesylated in yeast and mammalian cells (Clark et al., supra; Urano et al., J. Biol. Chem. 275:11198-206 (2000)) and shown to be localized to cell membranes as well (Clark et al., supra). Farnesylation of Rheb is critical for activity, as Rheb constructs lacking the CAAX domain could not complement yeast deficient for rheb (Urano et al., supra). One possibility is that when TSC1/2 is membrane-associated, it impedes Rheb function. Upon activation of PKB, disruption of the TSC1/2 complex may release inhibition of Rheb function.

TSC1/2 has also been implicated in amino acid signaling to TOR. Using S6K activity as a representation of TOR function, Gao et al. (Nat. Cell. Biol. 4:699-704 (2002)) showed that TSC1/2 is required for the normal reduction of S6K activity in response to amino acid starvation. Over-expression of Rheb consistently resulted in persistent S6K activity in the absence of amino acids. Remarkably, RNA interference studies demonstrated that Rheb was required for S6K phosphorylation, and presumably, activity. The data show that Rheb-mediated cell growth requires TOR, placing Rheb between TSC1/2 and TOR and thus as a downstream effector of insulin/PI3K signaling and nutrient sensing. Rheb has been implicated to regulate amino acid import in S. cerevisiae, but in a manner opposite of what would be expected of a growth-promoter. Rheb mutants had an increase in uptake of arginine and lysine (Urano et al., supra), suggesting that Rheb restricts amino acid import. Another interpretation of these data is that the increase in amino acid uptake is an indirect effect of losing Rheb. If Rheb normally stimulates nutrient import in S. cerevisiae, strains mutant for rheb may respond by upregulating alternative pathways.

Levels of Rheb mRNA are induced upon protein starvation and subsequently reduced upon refeeding. This agrees with findings that Rheb is rapidly induced following nitrogen starvation in A. fumigatus (Panepinto et al., supra). Overexpressed Rheb can still function in the presence of limiting environmental nutrients, leading to increased cell size in animals starved for protein and maintaining activation of S6K in cells cultured in the absence of amino acids. These results suggest that Rheb acts directly in promoting nutrient import. In S. pombe, Rheb has been shown to be required for cells to grow normally under limited amounts of nitrogen (Mach et al., supra). Together these data suggest that the induction of Rheb in response to nitrogen or protein starvation may be a means to mobilize limited resources and thereby maintain homeostasis under non-optimal conditions.

These results indicate that TOR is epistatic to Rheb. Rheb is, however, a proximal downstream component that recapitulates a cellular growth phenotype associated with hyper-insulin signaling. While tissue culture studies demonstrate that Rheb activates the TOR target, S6K, it is unlikely that S6K is the principle effector of Rheb-mediated growth. Over-expressed S6K failed to induce a cellular growth phenotype as seen with Rheb in starved animals (FIG. 7), and importantly, Rheb was able to promote overgrowth in animals mutant for S6K. Another target of TOR is 4E-BP, a translational repressor that becomes inactivated following phosphorylation by TOR. Flies null for 4E-BP are viable and fail to exhibit overgrowth phenotypes (Miron et al., Nat. Cell. Bio. 3:596-610 (2001)), making 4E-BP an unlikely candidate. Screens for revertants of Rheb-directed overgrowth will reveal the downstream effectors of Rheb (Miron et al., supra).

The previous examples are provided to illustrate but not to limit the scope of the claimed inventions. Other variants of the inventions will be readily apparent to those of ordinary skill in the art and encompassed by the appended claims. All publications, patents, patent applications and other references cited herein are hereby incorporated by reference.

1. A method for identifying a lead compound for diabetes drug development, comprising:
contacting a first aliquot of cells expressing a Rheb protein with a candidate compound under suitable conditions and for a period of time sufficient to affect Rheb activity;
measuring a parameter of the first aliquot of cells, the parameter associated with Rheb activity;
measuring the parameter in a second aliquot of control cells; and
comparing the measured parameters of the first and second aliquots of cells, wherein a change in the parameter is associated with an increase in Rheb activity.

2. The method of claim 1, wherein the Rheb protein is over-expressed and the parameter is cell size.

3. The method of claim 1, wherein the Rheb protein is over-expressed and the parameter is cell viability.

4. The method of claim 1, wherein the parameter is glucose uptake or utilization.

5. The method of claim 1, wherein the Rheb protein is human or Drosophila Rheb protein.

6. The method of claim 1, further comprising:
utilizing the candidate compound as a lead compound for diabetes drug development.

7. A method for identifying a lead compound for diabetes drug development, comprising:
contacting a candidate compound with Rheb protein under conditions conducive to binding of the compound to the Rheb protein;
detecting a resulting candidate compound-Rheb protein complex, and
determining whether the candidate compound increases or decreases Rheb protein activity.

8. The method of claim 7, further comprising:
utilizing the candidate compound as a lead compound for diabetes drug development.

9. The method of claim 7, wherein the Rheb protein is human or Drosophila Rheb protein.

10. The method of claim 9, wherein the Rheb protein is human Rheb protein.

11. The method of claim 7, wherein the candidate compound alters Rheb GTPase activity.

12. The method of claim 7, wherein the contacting is in cultured cells, and the stimulation of Rheb activity is detected by an increase in cell size or a prolongation of cell viability.

13. The method of claim 12, wherein the Rheb protein is over-expressed in the cultured cells.

14. The method of claim 7, wherein the contacting is in Drosophila larvae.

15. The method of claim 7, wherein the contacting is by administration of the candidate compound to Drosophila during eye development, and the stimulation of Rheb activity is detected by an enlarged eye phenotype.

16. The method of claim 7, wherein the Rheb protein is human Rheb protein expressed in Drosophila cells.

17. The method of claim 6, wherein the candidate compound increases glucose uptake or utilization.

18. A method for screening a library of candidate compounds to identify a lead compound for diabetes drug development, comprising:
contacting the candidate compounds with cells expressing a Rheb protein under suitable conditions and for a period of time sufficient to affect Rheb activity;
measuring a parameter of the contacted cells for a change in phenotype associated with Rheb agonist activity; and
determining whether the candidate compounds stimulate Rheb activity to identify a Rheb agonist.

19. The method of claim 18, wherein the measured parameter is cell size or cell viability.

20. The method of claim 18, wherein the measured parameter is the size of the eye in Drosophila.

21. The method of claim 18, wherein the measured parameter is glucose uptake or utilization.

22. The method of claim 18, measured parameter is GTPase activity.

23. The method of claim 18, wherein the Rheb protein is over-expressed in the cells.

24. The method of claim 18, further comprising:
utilizing the Rheb agonist as a lead compound for diabetes drug development.

25. A method for identifying a lead compound for drug development for a disease associated with abnormal cell growth, comprising:
contacting a first aliquot of cells expressing a Rheb protein with a candidate compound under suitable conditions and for a period of time sufficient to affect Rheb activity;
measuring a parameter of the first aliquot of cells;
measuring the parameter in a second aliquot of control cells; and
comparing the measured parameters of the first and second aliquots of cells, wherein a change in the parameter is associated with a change in Rheb activity.

26. The method of claim 25, further comprising:
utilizing the candidate compound as a lead compound for drug development for the disease associated with abnormal cell growth.

27. The method of claim 25, wherein the candidate compound inhibits Rheb activity.

28. The method of claim 25, wherein the Rheb protein is human or Drosophila Rheb protein.

29. The method of claim 25, wherein the measured parameter is cell size.

30. The method of claim 25, wherein the parameter is glucose uptake or utilization.

31. A method for screening a library of candidate compounds to identify a lead compound for drug development for a disease associated with abnormal cell growth, comprising:
contacting the candidate compounds with cells overexpressing a Rheb protein under suitable conditions and for a period of time sufficient to affect Rheb activity
measuring a parameter of the contacted cells for a change in phenotype associated with Rheb antagonist activity; and
determining whether a candidate compound inhibits Rheb activity to identify a Rheb antagonist.

32. The method of claim 31, further comprising:
utilizing the Rheb antagonist as a lead compound for drug development for the disease associated with abnormal cell growth.

33. The method of claim 31, wherein the Rheb protein is human or Drosophila Rheb protein.

34. The method of claim 31, wherein the measured parameter is cell size.

35. The method of claim 31, wherein the parameter is glucose uptake or utilization.

36. A non-human, transgenic animal over-expressing Rheb protein, wherein the animal has increased cell or organ size as compared with an animal not over-expressing Rheb protein.

37. The transgenic animal of claim 36, comprising human or Drosophila Rheb protein.

38. The transgenic animal of claim 36, wherein the transgenic animal is a primate, mammal, bovine, porcine, ovine, equine, avian, rodent, fowl, piscine, or crustacean.

39. The transgenic animal of claim 38, wherein the transgenic animal is a farm animal.

40. The transgenic animal of claim 39, wherein the farm animal is a chicken, cow, bull, horse, pig, sheep, goose or duck.

41. A transgenic, non-human animal over-expressing whose Rheb protein, wherein the over-expression results in increased size or growth rate of the animal.

42. A method for increasing the size or growth rate of a non-human, transgenic animal, comprising:
stably introducing into a genome of an animal cell a Rheb gene, whereby Rheb protein is over-expressed; and
producing an animal from the animal cell.
